(19) United States
(12) Patent Application Publication
(10) Pub. No.: US 2005/0079511 A1
(43) Pub. Date: Apr. 14, 2005

(54) Drug model explorer

(75) Inventors: Jacob W. Mandema, Atherton, CA (US); Michael J. Schwartz, Mill Valley, CA (US); Timothy Matthew Sheiner, San Francisco, CA (US); Jean-Max Vally, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pharsight Corporation, Mountain View, CA (US)

(21) Appl. No.: 10/773,767

(22) Filed: Feb. 6, 2004

Related U.S. Application Data
(60) Provisional application No. 60/511,602, filed on Oct. 14, 2003.

(51) Int. Cl. C12Q 1/68; G06F 19/00; G01N 33/48; G01N 33/50

(52) U.S. Cl. 435/6; 702/19

(57) ABSTRACT

Computer systems and methods facilitate exploring results of drug candidate modeling. In one embodiment, the software is configured to receive raw data simulated by a probabilistic model of clinical safety, tolerability, and efficacy of a drug candidate. Index information is extracted from the raw data and then referenced to generate a metadata file, the structure of the metadata file explicitly reflecting a hierarchical structure of the model. The metadata file is in turn used to convert the raw data into a binary file, the metadata file explicitly identifying locations within the binary file, of treatment scenario information types and output performance information types. The metadata file is also referenced to generate an interface configured to receive inputs from a non-expert audience, and in turn present relevant subsets of the binary file in a limited number of plot and tabular formats. By standardizing presentation and manipulation of data from different models, software and methods in accordance with the present invention facilitate meaningful interaction between a non-expert audience, and the complex abstract mathematical models predicting drug behavior. The heightened audience-model interaction afforded by the present invention in turn promotes uniform and consistent evaluation of modeled data in the process of drug development.

### CROSS-REFERENCE TO RELATED APPLICATION

The instant nonprovisional patent application claims priority from U.S. provisional patent application no. 60/511,602, filed Oct. 14, 2003 and incorporated by reference herein for all purposes.

### BACKGROUND OF THE INVENTION

The development of drugs is a lengthy and expensive process. In general, potentially efficacious compounds are first identified based upon their structure and/or properties exhibited during tests conducted in vitro. Next, those compounds exhibiting favorable properties in the laboratory are inserted into non-human organisms as drug candidates during pre-clinical testing.

In the next stage, drug candidates exhibiting favorable properties during pre-clinical testing are then subject to clinical testing in humans, first in small populations and then in larger populations. The expense of testing escalates with each stage, escalating particularly dramatically with the commencement of clinical human trials.

Typically, the clinical stage of drug development is divided into three phases. In the first phase (I), single and multiple dose escalation studies are performed in small groups of healthy volunteers to obtain pharmacokinetic data, safety data, and data on biomarkers related to the mechanism of action. About sixty percent of compounds entering phase I are passed on to the second clinical phase (II)

In phase II of clinical testing, multiple dose, dose ranging studies are performed in relatively small groups of patients to obtain clinical safety, tolerability, and efficacy data across a range of possible treatment options. About forty percent of the compounds entering phase II are passed on to the third clinical phase (III).

In phase III, pivotal safety and efficacy trials are performed in a large number of patients to support specific claims about the clinical benefits of a particular treatment strategy with the compound of interest. About seventy percent of the compounds that enter phase II make it to the next phase, which is submission of a new drug application (NDA) to the food and drug administration (FDA).

The process of deciding (1) which compounds to move to the next stage of development, (2) when to move a compound to the next stage, and (3) specific trials to complete in the next stage, is complex, requiring high-stakes decisions to be made with a significant amount of uncertainty.

On one hand, most drug candidates entering the clinical development process ultimately fail. Moreover, the costs of the drug development process (especially towards the later stages) is enormous. Thus one critical aspect of the decision-making process is to halt, as early as possible, testing of candidates having a low probability of success.

On the other hand, due to the tremendous return on a drug that actually makes it to the marketplace, there is the tendency to continue developing compounds that have some probability to succeed. Furthermore, because of the limited and fixed patent life of drug compounds, there is significant pressure to bring potentially successful candidates to the marketplace as fast as possible.

One particularly critical task of early stages (pre-clinical and phases I-II) of clinical drug development, is to provide sufficient understanding of the probability that a potential drug candidate is actually a marketable drug product. Such marketable drug products offer sufficient benefit over other treatment options, to warrant investment in the pivotal phase III trials. Early development of a drug candidate should also provide sufficient understanding of both the optimal treatment strategy, and the target patient population, for those compounds moving forward in the drug development process.

In practice, this amounts to answering a number of questions as quickly as possible regarding the drug's likely clinical safety, tolerability, and efficacy profile emerging from early development trial data. Examples of such questions include, but are not limited to:
1. What beneficial effects are likely to be demonstrated by the drug candidate?
2. What adverse events are likely to arise from the use of the drug candidate?
3. What is patient tolerability for the drug candidate?
4. How are clinical outcomes for the drug candidate related to dose?
5. How are clinical outcomes for the drug candidate related to characteristics of the patient population?
6. How does the drug candidate compare with potential competitors based on the above criteria?

Conventionally, it has proven difficult to answer the above questions for a number of reasons. For example, in early drug development relatively little clinical outcome data may exist for the drug candidate. This limited availability of hard data may influence, with high variability, decisions made regarding the drug candidate.

Moreover, while non-clinical outcome data on the drug candidate may exist based upon pre-clinical studies, early clinical safety studies, and biomarker studies, the relationship of this data to actual clinical outcomes may be uncertain. This uncertainty can again grossly influence decisions made regarding the future of a particular drug candidate.

Engaging in consistent and methodical decision-making regarding a particular drug candidate may further be complicated by the location of data regarding the candidate compound and its competitors. For example, relevant data regarding a drug candidate compound and its competitors may be stored in a variety of public and private databases having different goals, origins, and structures.

Finally, early clinical data that has been found to exist may not be directly comparable owing to differences in methodology utilized in collecting the data. For example, existing pre-clinical data may have been collected utilizing animal studies. The results of these studies are not directly comparable to clinical outcome studies, but contain relevant information regarding the potential clinical safety and efficacy profile.

Similarly, early clinical biomarker studies (phase I) may be completed in healthy volunteers. Both the endpoint and patient population are not directly comparable to the clinical outcome studies, but the biomarker trial results contain important information on potential clinical safety and efficacy. Similarly, clinical outcome studies on competitors may have used different endpoints and patient populations, rendering any direct comparison between the candidate and its competitor a difficult task.

As a result of difficulties posed by these considerations, the above-listed questions regarding early stage clinical drug development are conventionally answered by focusing upon several independent representations of the characteristics of drug candidate compounds, for example summaries of specific results from independent trials and experiments. These independent pieces of information are circulated and discussed to support decisions on the continued development of the compound.

While providing some information regarding a drug candidate, these independent representations do not provide a comprehensive response to the critical questions arising in early clinical stages of drug development. Moreover, the representations do not quantify the risk involved in relying upon them for decision-making.

Accordingly, there is a need in the art for systems for modeling the behavior of drug candidates that integrates the relevant public and proprietary data from different sources, type and structure, spanning discovery to clinical development into a probabilistic model of the compound's clinical safety, tolerability and efficacy profile in relation to the compound's competitors.

There is a need in the art for systems to make the knowledge contained in these drug models broadly accessible to the clinical development organization so that the members of this organization can explore the knowledge, summarize the knowledge, communicate the knowledge and make decisions about the development on basis of this knowledge.

### SUMMARY OF THE INVENTION

Computer systems and methods facilitate exploring results of drug candidate modeling. In one embodiment, the software is configured to receive raw data simulated by a model of clinical safety, tolerability, and efficacy of a drug candidate. Index information is extracted from the raw data and then referenced to generate a metadata file, the structure of the metadata file explicitly reflecting a hierarchical structure of the model. The metadata file is in turn used to convert the raw data into a binary file, the metadata file explicitly identifying locations within the binary file, of treatment scenario information types and output performance information types. The metadata file is also referenced to generate an interface configured to receive inputs from a non-expert audience, and in turn present relevant subsets of the binary file in a limited number of formats. By standardizing presentation and manipulation of data from different models, software and methods in accordance with the present invention facilitate meaningful interaction between a non-expert audience, and the complex abstract mathematical models predicting drug behavior. The heightened audience-model interaction afforded by the present invention in turn promotes uniform and consistent evaluation of modeled data in the process of drug development.

A modeling methodology may develop a probabilistic model profiling clinical safety, tolerability, and efficacy of a candidate drug compound. The model may integrate relevant data spanning the period from initial discovery to clinical development, the data originating from public and private sources and exhibiting different structures. A non-expert audience utilizing software methods in accordance with the present invention may efficiently explore information resulting from this modeling.

In order to provide rapid access to information contained in the model, a large database is simulated containing samples of the probability distribution of each endpoint represented in the model, as a function of input variables. Examples of such input variables include, but are not limited to, dose, dose frequency, time, patient characteristics, assumptions, and other variables impacting behavior of the drug candidate.

The software receives the simulated data and generates a corresponding metadata file identifying the location of different types of information present therein. The software specifies a graphical user interface allowing non-experts to explore, summarize, and communicate the information contained in the drug models. The software user provides input to the software based upon a limited but comprehensive set of input parameters, for example endpoints, controllable variables, and uncontrollable variables. Referencing the metadata, the software extracts from the binary file those subsets of data relevant to the user inputs, performing additional analyses if necessary.

This corresponding output is presented to the user in a number of plot and tabular formats. The software thus facilitates non-expert interaction with complex drug behavior models, streamlining the drug development process by providing decision-makers with a standardized framework for characterizing drug behavior across different candidates, across different models, and in relation to different competitors.

An embodiment of a method of representing performance of a drug candidate in accordance with the present invention, comprises, receiving raw data generated by a model of drug candidate behavior, the raw data comprising index information, treatment scenario input information types, and corresponding output performance information types. Index information is extracted from the raw data. The extracted index information is referenced to generate a metadata file, a structure of the metadata file explicitly reflecting a hierarchical structure of the model. The metadata file is referenced to convert the raw data file into a binary file, the metadata file explicitly identifying locations of treatment scenario information types and the output performance information types within the binary file. A user interface is generated from the metadata file, the interface comprising a menu of input variables. The menu is presented to a user. A user-selected input is received at the interface. The interface is caused to reference the metadata file and the binary file to identify a subset of the binary file relevant to the user-selected input. The data subset is presented in one of a select type of presentation formats at the interface.

An embodiment of a computer system in accordance with the present invention, comprises, a processor and a memory storing code to operate the processor. The code comprises a parser module configured to receive raw data output by a model of drug candidate behavior, and to generate a metadata file encoding outputs and related inputs of the model based upon index information extracted from the raw data. The code also comprises a data transfer module configured to convert the raw data into a binary file organized to match a structure encoded in the metadata file. The code further comprises a graphic user interface configured to present a menu of input variables to a user, to receive inputs selected by the user, to reference the metadata file and the binary file to identify a subset of the binary file relevant to the selected inputs, and to present the data subset in one of a select type of presentation format.

These and other embodiments of the present invention are described in more detail in conjunction with the text below and attached figures.

### BRIEF DESCRIPTION OF THE DRAWINGS

FIG. 1 shows a simplified schematic diagram depicting the role of the Drug Model Explorer (DMX) software in the drug development process.

FIG. 2 shows a simplified schematic diagram of a conventional systems for evaluating suitability of a drug candidate as a commercial product.

FIG. 3 shows a simplified schematic diagram of the use of modeling in accordance with embodiments of the present invention, to evaluate suitability of a drug candidate as a commercial product.

FIG. 4 shows an automated system for performing the drug evaluation function shown in FIG. 3.

FIG. 5 shows a simplified schematic diagram of user inputs and outputs to one embodiment of a DMX software program in accordance with the present invention.

FIG. 6 shows a simplified depiction of generic fields of one embodiment of a graphic user interface of the DMX software program.

FIGS. 7A-N shows specific screen shots of one graphic user interface of the DMX software.

FIG. 8 is a schematic illustration of a computer system for use in accordance with embodiments of the present invention.

FIG. 8A is an illustration of basic subsystems the computer system of FIG. 8.

FIG. 9 is a simplified schematic diagram showing operation of an embodiment of the software in accordance with the present invention.

FIG. 10A is a simplified schematic diagram showing generation of output from a model based upon provision of specific input variables.

FIG. 10B is a simplified schematic diagram showing generation of metadata and corresponding binary output files from raw model output having an arbitrary format.

### DESCRIPTION OF THE SPECIFIC EMBODIMENTS

The Drug Model Explorer software (“DMX software”) in accordance with embodiments of the present invention, comprises a technology platform enabling pharmaceutical companies to adopt an integrated, quantitative, model-based approach to decision-making regarding clinical drug development. The DMX software enhances understanding of possible clinical potential and limitations of a drug relative to competitors at any point during development, and distributes that understanding across a project team and decision-makers. Users of the DMX software will be able to compare the probability distribution for different endpoints such as biomarker, efficacy, safety, and tolerability, for different treatment strategies, for different patient populations, and for different competing products.

In accordance with one particular application, the DMX software may be utilized to facilitate decision-making regarding clinical development programs for particular drugs. Specifically, where models have been created of the potential product profile of drugs under development, the DMX software can be employed to support critical decisions in the development process for these drugs. Such models supporting these decisions quantify the probability distribution of clinical outcomes such as efficacy, safety, tolerability, and biomarkers, as a function of treatment strategy, treatment duration, and patient and disease characteristics. The models provide an integrated view of the likely clinical behavior of the drug given the current state of knowledge.

The DMX software application allows clinical project team members to understand and interactively explore the knowledge contained in these drug models to support ongoing decision-making. The DMX software is a visualization and communication tool that provides access to the expected product profile. As such, the DMX software is intended to enhance understanding of a drug's likely clinical potential and limitations relative to competitors at any point during development, and to more broadly distribute that understanding across a project team and senior decision makers.

In accordance with one embodiment, the DMX software may be deployed in the development of a drug indicated for disease-modifying treatment of osteoarthritis and rheumatoid arthritis. Among the important questions facing the development of this drug include the strength of the drug-exposure relationship for biomarker, the strength of the drug-exposure relationship for clinical endpoints, and whether (and if so, how) to develop an extended release drug formulation for one or more indications.

The DMX software technology may be utilized to address other issues such as the appropriate target population for each indication, the optimal dosing regimen, the optimal formulation, for example for immediate release vs. extended release, or some combination of the two approaches. Other issues that may be addressed utilizing the DMX software include, but are not limited, to the likelihood of clinical benefit and risk vs. major competitors. The DMX software technology may also be utilized to enhance the contribution of modeling and simulation to project team-level decision making.

The DMX software is designed as a visualization and communication tool to provide access to the expected product profile and to make drug and disease modeling results accessible to project team members and decision-makers. Model outputs may be interrogated and viewed by project team members via an intuitive user interface.

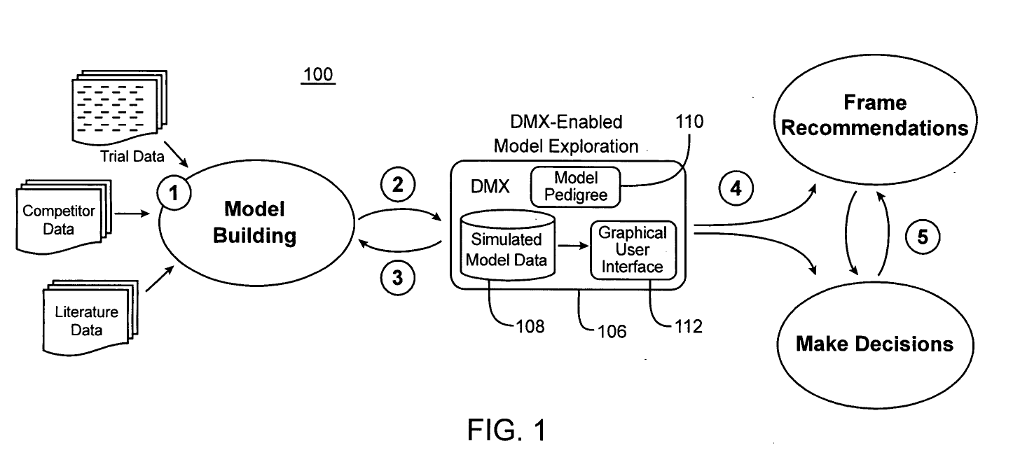

FIG. 1 shows a simplified schematic diagram depicting the role of the DMX software in the drug development process. FIG. 1 allows visualization of the interplay between model builders, DMX software users, and decision-makers. First step 1 of process 100 comprises drug modeling & model building. In this first step, the data analyst constructs the models on the basis of available information such as clinical trial data, competitor data, and literature data. The development team presents the analyst with requests for initial views.

In second step 2, results from the drug models are loaded into the DMX software 106. The DMX software may be populated with a simulated database 108 containing the probability distribution of a summary statistics such as mean or fraction of patients above a target, for efficacy, safety, or other endpoints as a function of specific model inputs, such as treatment options (drug, dose, dose frequency, etc.), patient populations, and assumptions. Database 108, and its associated metadata, characterizes the ‘space’ that can be explored by the DMX software. The analyst may also populate the DMX software with an overview of the model pedigree 110 (documentation on source data, validation, conclusions).

The DMX software also includes a graphic user interface (GUI) component 112. The GUI may allow the DMX software user to graphically view the expectation and uncertainty (percentile uncertainty bands) of selected endpoints as a function of continuous input variables (xy-plot) and discrete input variables (box-plot). This information can be viewed as a table.

The DMX software may also allow a user to view (in both graphic and tabular form) the expectation and uncertainty of the difference in an endpoint between one set of input variables and another set of input variables, for example a reference. A user may select and vary 1) endpoints that are displayed, 2) input variables for which the endpoints are displayed, and/or 3) the reference (comparators) against which another input is compared.

The DMX software allows a user to view multiple endpoints for multiple combinations of input variables and multiple references. The DMX software allows a user to partition endpoints (or difference if contrast is selected), in categories such as inferior, equivalent, and superior. The probability of falling in these categories for multiple combinations of input variables and multiple contrasts may be viewed. The value of a certain input variable (such as dose) required to achieve performance in a certain category may also be viewed. The selected input parameters for construction of a Clinical Utility Index (CUI) may be viewed and changed. Pre-defined views presented by the DMX software may be saved, restored, and shared.

In the third stage 3 of FIG. 1, the drug development team may explore the model utilizing the DMX software, resulting in model refinement and feedback. In this stage, multi-disciplinary team members come together and use the DMX software to explore both graphically and in tabular form, likely clinical potential and limitations of a compound, in order to improve communication and to inform decision-making. Utilizing the DMX software, team members are able to actively compare knowledge contained in drug-disease models, allowing them to explore, for example, precision in dose-response for different endpoints, different treatment regimens, patient populations, or different competing products.

As model-building and decision making are interactive processes, new questions will arise, assumptions can change, new data can become available, or certain questions will become obsolete. In one or any of these evolving landscapes, the DMX software can facilitate updating the model and/or publication of a new simulation database for team exploration.

Specifically, the fourth step 4 of FIG. 1 shows the DMX software used to frame insights and recommendations for decision-making. Users of the DMX software may want to preserve certain DMX software views that capture the main insights from their exploration of the model, in order to support the team recommendations for further development.

In the fifth step 5, the DMX software can be used to effectively communicate, both graphically and in tabular form, the effects of the drug relative to internal and external competitors. Such real-time exploration of the current knowledge of effects may enhance the ability to make informed decisions regarding the development strategy for the drug. Senior stakeholders will be presented with uniform and consistent views summarizing the exploration underlying decision recommendations, but will have the option to modify certain choices themselves, using the summary views as a starting point.

Once the DMX software has been utilized to present information regarding drug candidates, this information can be utilized by the decision-making team to move forward with additional laboratory or clinical testing.

The role played by the DMX software in accordance with embodiments of the present invention may be contrasted with conventional approaches to drug design. As explained in detail below, such conventional approaches are typically dominated by the role of the human expert in creating models of drug candidate behavior, and then presenting those results to a non-expert audience for exploration.

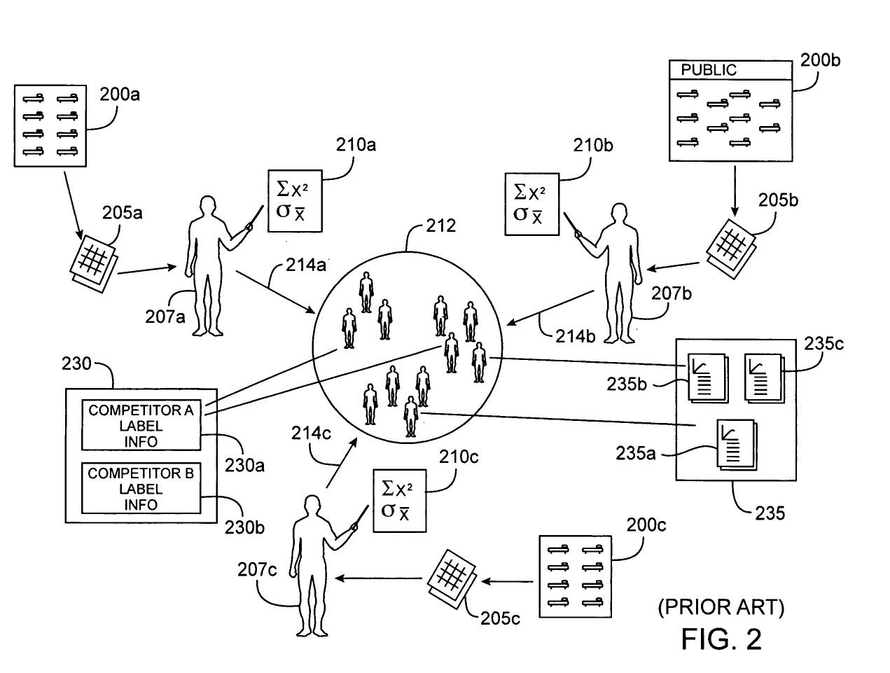

Specifically, FIG. 2 shows a simplified schematic view of a conventional system for investigating suitability of drug candidate as a commercial product. System 201 comprises clinical studies 200a-c involving the drug candidate.

Studies 200a-c are generally run under different conditions, so that the corresponding study results 205a-c are not directly comparable. Examples of parameters which may differ between different studies include, but are not limited to, numbers of subjects, treatment drug, treatment dosages, dosage patterns (i.e. number of times per day), length of treatment, population characteristics, length of study, schedule of recorded measurements, number of recorded measurements, location of study, laws governing collection of information, the study sponsor, and the particular organization and/or individuals administering the study.

Different human experts 207a-c analyze study results 205a-c respectively, producing summaries 210a-c. In practice, while one or more human experts 207a-c may be a single individual, it is also likely that they will be several individuals.

One or more of summaries 210a-c corresponding to studies 200a-c may be of the same or different types. For example, a summary may be based solely on statistical analysis of the study results, or they may be in the form of pharmacokinetic-pharmacodynamic (PK-PD) models based on the study results. Each summary will refer to, and be based on data from, the relevant study 200a-c, without reference to other studies or data sources.

Experts 207a-c present the summaries 210a-c to the audience 212, which may comprise experts and non-experts. For purposes of this patent application, the term “non-expert” refers to an individual lacking formal training in both pharmacology and statistics, for example a business professional invested with the responsibility of deciding whether or not to move forward with full clinical testing of a drug candidate.

In addition to clinical study summaries presented by experts, members of the decision-making team may also be exposed to other sources of information such as relevant scientific literature, 235a-c, and publicly available FDA labeling information on competitive compounds, 230a-b. However, members of the decision-making team may not have been exposed to the same additional information, or, for any number of reasons, may not have interpreted that additional information in the same way.

Audience 212 is charged with responsibility for developing a consensus view on behavior of the drug candidate. Audience 212 is also charged with making a recommendation regarding if and how to proceed with commercial development of the candidate.

The conventional decision-making process referred to in connection with FIG. 2 offers at least three significant inefficiencies. First, the summaries of clinical studies produced by various experts are the primary source of information upon which audience 212 may base its decisions. However, as apparent from FIG. 2 and the above written description, there is often a great deal of information available from other sources that is relevant to understanding the activity of a given drug candidate. The conventional process shown in FIG. 2 does not ensure that the decision-making team is exposed to this additional information in a uniform and accountable manner.

A second inefficiency of the conventional drug development decision-making process just described is the failure to integrate the different information sources. In considering the overall behavior of a drug candidate, and its likely value as a drug product, each member of the audience must perform an internal integration process comparing the results of the different studies and any additional sources of information previously encountered.

This process of internal integration by the audience is highly subjective, and depends upon appropriate factors as personal experience and intuition, and also potentially upon inappropriate factors such as pre-disposed attitudes, internal political affiliations, and differing levels of exposure to additional available information. As a result of the subjective nature of the study integration process, each member of the audience is likely to have a different opinion regarding the behavior and likely value of the drug candidate.

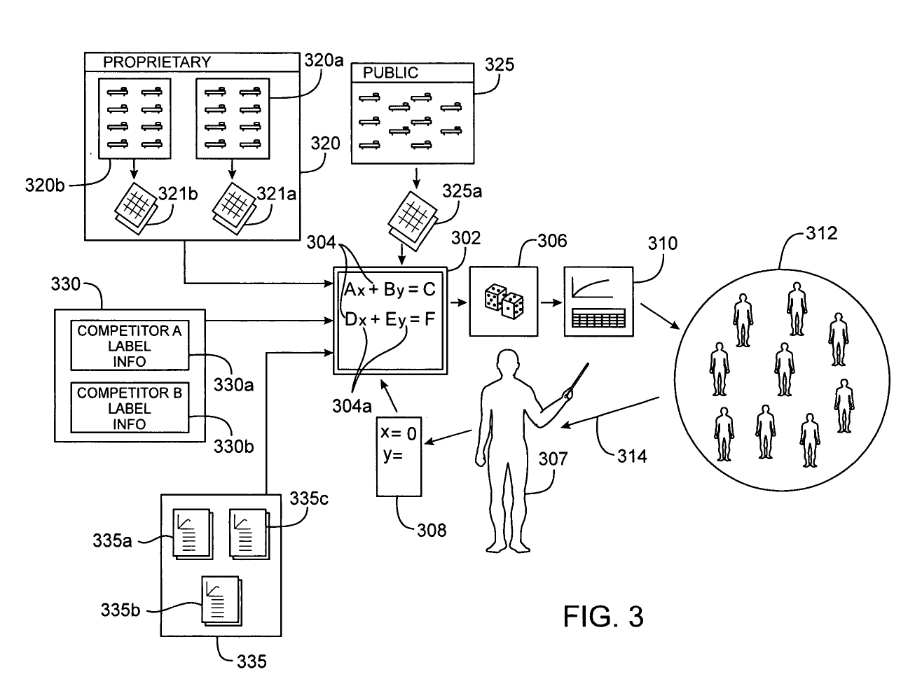

FIG. 3 shows a simplified schematic for a system, referred to herein as the “DMX Methodology”, which improves upon the conventional drug discovery decision-making system represented in FIG. 2. System 301 comprises a model 302 comprised of multiple equations, 304, with each equation having a plurality of terms, 304a.

Model 302 is constructed by human expert 307 based upon knowledge of the fields of physiology, pharmacology, and statistics, and information known about a drug candidate. Specifically, human expert 307 constructs model 302 by researching and integrating all sources of information relevant to the drug candidate.

Sources of information upon which model 302 may be constructed, include the group of proprietary clinical studies 320 conducted during development of the drug candidate. This group may consist of multiple individual studies 320a and 320b yielding results 321a and 321b, respectively. These items correspond directly with the conventional studies 200a and 200c and results 205a-c of FIG. 2.

FIG. 3 shows that sources of data other than clinical studies may be utilized in constructing model 302. Examples of such additional data sources include, but are not limited to, public studies 325 sponsored by universities, not-for-profit organizations, municipal bodies, and government agencies. FIG. 3 shows that yet another source of data for constructing models is the publicly-available labeling information 330 for drug products required by the Food And Drug Administration (FDA), whose method of action is deemed relevant to the drug candidate by the human expert 307. Still another data source may be scientific literature 335 deemed relevant to the drug candidate by the human expert 307.

Model 302 is thus constructed utilizing many known sources that provide data relevant to understanding the compound, thereby integrating as much information as possible known about the compound.

One aspect of this integration, is that the assumptions used to combine these non-uniform sources, are represented in the model with a quantitative assignment of their certainty. Specifically, particular sources of drug candidate performance information may be more reliable than others. For example, clinical study results may be particularly reliable if based upon a large study size. Such data used to construct the model may therefore have a stronger influence on model certainty value than information from other, less reliable sources. Other factors which may be considered in determining reliability of sources of information regarding a drug candidate include, but are not limited to, the reliability of investigators, the design of a clinical study including consideration of blinding, randomization, and appropriate controls, the antiquity of the study, and the source of the study, for example whether or not it is from a peer reviewed journal.

A result of the integration of information sources in accordance with embodiments of the present invention, is that the model output is probabilistic in nature. A given set of inputs produces an estimate both of the expected modeled effect and the likelihood that the expectation is true. In other words, the output of the model is a probabilistic distribution of effect.

An additional feature of the model is that owing to its mathematical form, the model can output predictions from input conditions lacking actual clinical data. Thus if clinical studies were conducted with a compound utilizing doses of 200 mg and 300 mg, the model could output distributions accurately reflecting the actual measured effects at those dosages. Moreover, the model could also output distributions reflecting the expected effect for a dose of 250 mg, for which no actual clinical data existed.

Based upon the input conditions 308, model 302 produces output 310 via simulation step 306. Output 310 predicts the effects likely to be observed under the conditions input, which, as stated above, may or may not be the result of actual clinical studies. This aspect of the present invention is thus unlike the conventional study summaries of FIG. 2, which represent only behavior of the candidate in the context of actual study conditions.

Output 310 may also include a quantification of uncertainty associated with the predictions, based upon the certainty values originally assigned or otherwise derived from source data utilized in constructing the model. This aspect of the present invention is thus also unlike the conventional study summaries of FIG. 2, which generally do not explicitly address uncertainty, leaving this determination to the subjective intuition of the modeling expert and/or audience members.

Simulation by the model is performed in conjunction with a computer in one or two steps. In accordance with one embodiment of the present invention, human expert 307 may ask model 302 to generate an output 310 describing variation in drug candidate behavior for only an individual patient

In the first phase, a number of hypothetical individuals predetermined at the discretion of the human expert 307, are created. Then, for a given effect and treatment scenario (i.e. drug candidate dose, frequency, and formulation), model 302 is operated for each hypothetical patient to calculate the resulting effect of the drug candidate in each individual. Thus at the conclusion of this first simulation phase, the model will output the predetermined number of different effect values for the specified treatment scenario. These values form a distribution amenable to analysis to provide statistics predicting variation in patient response to a given treatment scenario. Where patient-level simulation is the goal, this output may be provided directly to the DMX software, without further manipulation.

More commonly however, the goal of modeling is to communicate uncertainty of response to the drug candidate in a patient population. In such an application, it is desired that the model provide a probabilistic distribution of a summary statistic representing population response, for example a mean value of patient response.

Model 302 is constructed based on the pharmacology of the drug candidate with respect to an individual patient. Accordingly, producing population-level data according to the DMX methodology requires a second simulation step.

Specifically, this population-level data may be simulated by repeating the entire patient simulation process a second predetermined number of times, using the first predetermined number of patients for each iteration. In this second phase, however, a single statistic is calculated and retained from each distribution of the response values for each of the predetermined number of simulated patients. The resulting distribution of these statistic values reflects uncertainty about the true model used in the first step.

As with the first simulation phase, the end result of the second simulation phase will be a distribution of the first predetermined number. However, the distribution resulting from the second phase will comprise a statistic (such as a mean) derived from the second predetermined number of previous distributions of patient response, rather than the responses for the first predetermined number of different patients. For a highly certain model, the replications will closely resemble each other, and the recorded statistics will exhibit a narrow distribution. For a highly uncertain model, the replications will vary considerably, and the recorded statistics will exhibit a broad distribution. Presenting statistics based on this second distribution is another possible kind of output 310.

Expert 307 presents either form of output 310 to audience 312, which may comprise both experts and non-experts and is identical to audience 212 in FIG. 2. Along with presenting the output, expert 307 must also explain to audience 312 the assumptions underlying construction of the model—and the particular input conditions to the model upon which the representation is based.

Audience 312 receives the output and corresponding input and assumption information from expert 307. Audience 312 may then seek to investigate the effect upon this representation, of changing the input to the model and/or the assumptions of the model. In order to accomplish this task, audience 312 must communicate desired changes 314 to human expert 307, who in turn must again translate the changed inputs into different numerical values, and re-run the simulations.

Returning to model 302, it may be understood that this model improves on the prior art in at least three important ways. First, the model is constructed based upon all the information currently known about a drug candidate and the uncertainty associated with each piece of information. The model can be validated against this information to demonstrate its ability to accurately reflect actual clinical results.

Second, model encodes in an explicit and accountable way, the precise assumptions that the human expert 307 has brought to bear in its development. Thus, group decision-making process is facilitated because assumptions are exposed for discussion, modification and the development of consensus.

Finally, model 302 improves upon the prior art by permitting what-if analysis to be performed. Unlike the conventional clinical study summaries 210a-c of FIG. 2 which represent the drug candidate's effects in the context of the actual conditions studied, model 302 can accept input conditions 308, that were not actually studied. Via simulation step 306, the model can produce output 310 that predicts effects likely to be observed under those conditions, together with a precise statement of the uncertainty associated with those predictions.

As is apparent from the above written description, the audience/model feedback mechanism of the conventional system is dominated by the human expert. The human expert must translate abstract pharmacological concepts relevant to the audience into concrete mathematical relationships relevant to the model. This translation process not only slows audience/model feedback, but also creates a distance between the audience and the model, so that the audience may not develop a deep understanding or intuition about the drug candidate's behavior as predicted by the model.

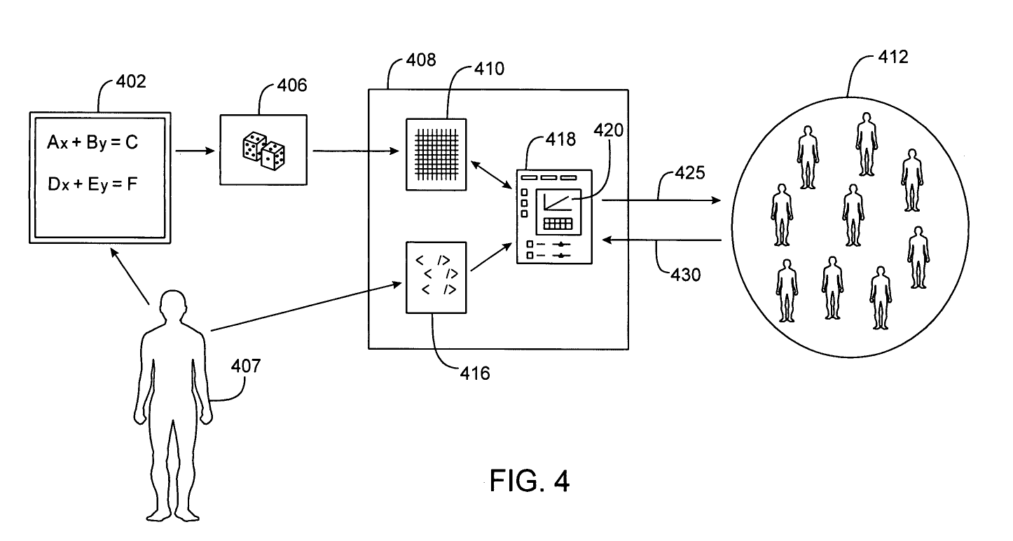

FIG. 4 shows a simplified schematic of a method for automating a portion of the DMX Methodology described in FIG. 3. This automation method is referred to in this patent application as the “DMX software.” The automation method as represented by entity 408 consists of a large binary data file, 410, a metadata file, 416, and software code which creates a graphical user interface 418 by which users can produce output 420.

The binary file 410 is a computer readable form of a large, n-dimensional hypercube of numbers, on the order of 1×10⁸ for larger simulated patient populations which is saved as the binary file 410. The numbers in the file are organized such that the set of unique input values producing a particular output distribution can be used to locate within the file, the corresponding set of numbers representing the distribution effect for a particular clinical endpoint.

The binary file 410 is created by human expert 407 by executing a large number of simulation runs 406 using model 402. Here expert 407 and model 402 are identical to expert 307 and model 302 of FIG. 3. Thus model 402, and the methodology by which it is developed, maintains the advantages over the prior art (FIG. 2) described in the text accompanying FIG. 3.

The simulation step 406 is similar to simulation step 306 of FIG. 3 with at least one significant difference. In the description of the DMX Methodology accompanying FIG. 3, it was indicated that the simulation step would calculate a distribution for a particular effect given a particular set of input conditions. However, in simulation step 406 of FIG. 4, expert 407 sets up simulation runs to calculate all modeled effects for a large number of combinations of different input conditions. In other words, the result of simulation step 406 is to produce data that describes completely a portion of the multi-dimensional space defined by all possible states of the model inputs and all possible resulting effects.

DMX software 408 is configured to receive the large file resulting from the simulation step 406 to permit the audience 412 to explore and visualize the different treatment response information contained therein without having to work through a human expert intermediary.

Specifically, DMX software 408 is configured to receive the file output from model 402 via simulation step 406, and to generate therefrom a binary file 410 and corresponding metadata file 416 allowing interpretation of binary file 410. The metadata file, also produced by human expert 407, is a crucial component of the system. One function of metadata file 416 is to provide the interface logic of the software with an index to the binary data file. In other words, metadata 416 unambiguously explains the meaning of every number comprising the output binary file.

A second role played by metadata file 416 is to dynamically configure the display component of the software. Metadata file 416 can be thought of as a set of instructions describing the hierarchical structure of the model displayed to audience through the software.

Finally, the metadata file 416 instructs the software which binary data file is associated with the metadata, so that the software may load that data.

The modular design of the metadata component means that a single instantiation of the software is able to display results from any model having metadata and binary data. Thus human expert 407 can produce a data set comprising binary data 410 and metadata 416 for any number of different drug candidates, and any member of audience 412 who has access to 418, the graphical user interface component of the DMX software, can visualize and explore that data. In the context of drug development, the DMX software thus offers a general solution for a multitude of drug development programs, as the interface/data structure is not specific to a particular drug modeling program.

By virtue of its multi-functional role, the metadata serves as the link between the audience, the model, and the mass data produced by the model. The metadata component provides the DMX software with the ability to represent model inputs to the audience in the input component. When the audience configures the input component to specify interest in a particular input scenario, the structure contained in the metadata is also the key reference for the DMX interface logic to determine those specific locations in the binary data file to be used to calculate the appropriate output.

Graphic user interface 418 of DMX software 408 receives the binary and metadata files, facilitating display of modeled data to audience 412. Specifically, after reading metadata file 416, DMX software 408 draws itself upon the screen of the user's computer as graphical user interface 418, using the information in the metadata file to create the appropriate input controls carrying the appropriate labels necessary to give the user access to all of the population and treatment scenarios stored in the binary data. By selecting and editing values of the input controls 418, audience 412 is able to directly produce output 420 that appears in the output display component of the DMX Interface.

As shown in FIG. 4 and explained above, the DMX software creates the experience of a direct and immediate communication path, represented as flows 425 and 430 between audience 412 and model 402. While in fact, this connection is not truly as direct as the experience feels, the hidden components become irrelevant to the audience's ability to freely explore and discuss the drug candidate's effects as shown in output 420.

In turn, this easy exploration of a shared information context facilitates the group processes involved in drug development decision-making.

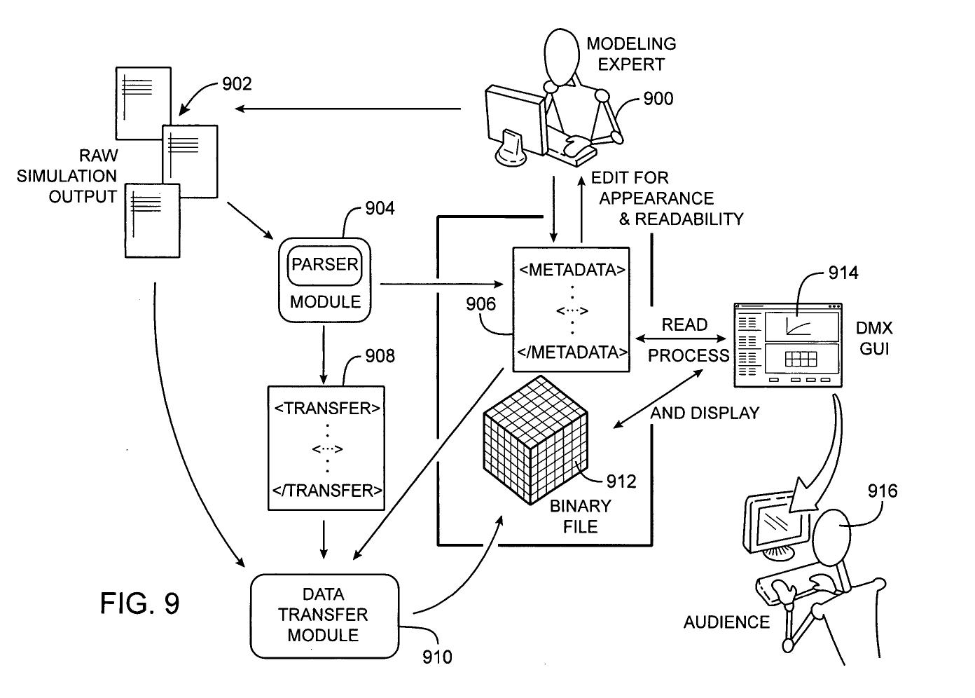

FIG. 9 shows a schematic diagram illustrating in greater detail operation of the DMX software. After developing a model, modeling expert 900 uses it to run simulations producing a collection of files 902 comprising raw simulation output data. Raw output data files 902 may exhibit a format corresponding to that of any number of software programs utilized for statistical analysis and modeling, including but not limited to software developed by SAS Institute Inc. of Cary, N.C., S-PLUS software developed by Insightful of Seattle, Wash., EXCEL software developed by Microsoft Corp. of Redmond, Wash., and various internally-developed software programs proprietary to specific organizations.

Modeling expert 900 then provides raw simulation output files 902 to parser module 904 of the DMX software. Parser module 904 reads files 902 and produces two separate output files 906 and 908.

The first file output by DMX Parser Module 904 is the Metadata file 906. Metadata file 906 encodes a hierarchical structure of the model (i.e. the outputs and related inputs) that is implicit in the structure of the raw simulation files 902. This encoding is defined within the Metadata file in terms of labels used in the raw simulation output files.

The second file output by DMX Parser Module 904 is the Transfer file 908. Transfer file 908 identifies those raw simulation output files containing the data for each component of the model structure encoded in Metadata file 906.

Modeling expert 900 then provides the Transfer file 908 and the Metadata file 906, along with the raw simulation output files 902, to data transfer module 910 of the DMX software. Data transfer module 910 comprises software which converts the multiple raw simulation output files into a single binary file 912.

Binary file 912 is organized to match the structure encoded in the metadata file 906. In the view of FIG. 9, the binary file is depicted as having a three dimensional geometry for ease of illustration. However, embodiments in accordance with the present invention are not limited to generating a binary file exhibiting a geometry having this, or any other, number of dimensions.

The DMX software uses the resulting binary and metadata files 912 and 906 to produce graphic user interface (GUI) 914, through which audience 916 can investigate the conclusions of the modeling and simulation work done by the modeling expert 900. Because the text in Metadata file 906 is used to produce the labels of GUI 914, modeling expert 900 may edit some of the metadata text for clarity before supplying metadata file 906 and its companion binary file 912, to audience 916.

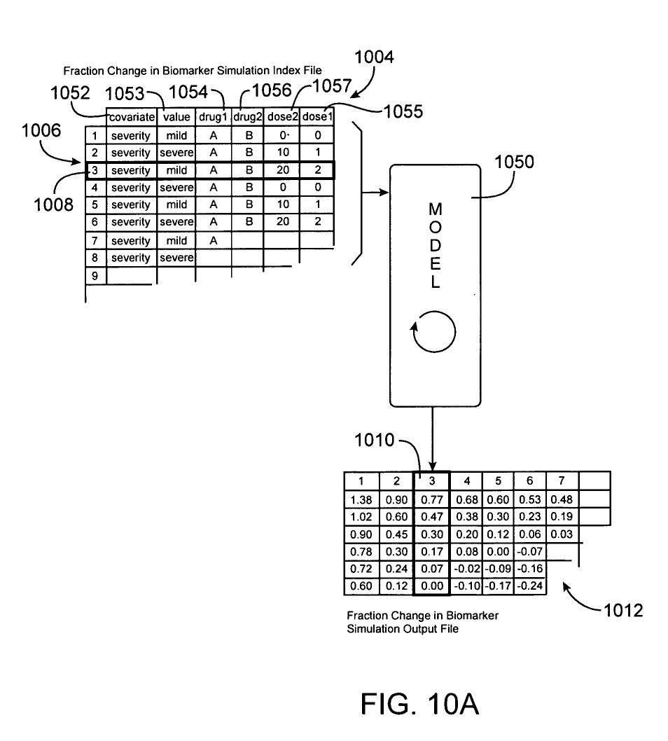

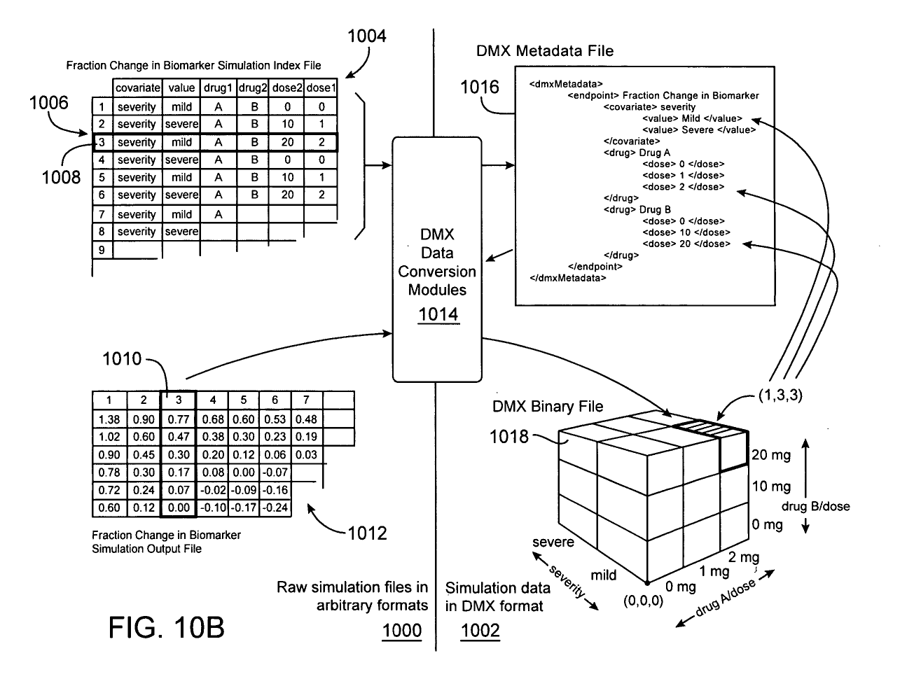

The relationship between the modeling of drug candidate performance, and operation of the DMX software, is illustrated and described in connection with FIGS. 10A-B. FIG. 10A shows a simplified schematic diagram illustrating operation of drug candidate performance model 1050. Model 1050 is configured to receive as inputs an variety of treatment scenarios shown in simulation index file 1004. Each row of index file 1004 identifies a particular treatment scenario comprising six variables.

A first treatment scenario variable 1052 is called covariate and has only a single value, “severity”, which corresponds to disease state. A second variable 1053, dependent upon variable 1052, is called “value”, and may be either “mild” or “severe”.

A third treatment scenario variable 1054 is the identity of a first drug (“drug1”) utilized to treat the disease. Here, the variable “drug1” can only have the value “A.”

A fourth input variable 1055 (“dose1”), dependent on variable drug1, corresponds to the dose of the first drug. This variable may have a number of values, but only three possible doses (0, 1, or 2) of drug “A” are shown in FIG. 10A.

A fifth treatment scenario variable 1056 (“drug2”), corresponds to the identity of a second drug utilized to treat the disease. In this example, this variable may have only the single value “B.”

Finally, variable dose2, 1057, represents the dose of the second drug. Again, this variable may have a number of values, but only three possible doses (0, 10, or 20) of drug “B” are shown in FIG. 10A.

As described in detail above, model 1050 receives inputs 1052-1057, and generates therefrom output file 1012 containing columns of numerical values corresponding to simulated drug candidate performance based upon a particular treatment scenario. Output file 1012 thus represents the result of multiple calculations by the model.

As previously described, where the model is employed to simulate response of an individual patient, these multiple calculations generate columns of numbers representing output based upon variation in patient characteristics. In the more useful and common instance where the model simulates response of a patient population, the multiple calculations generate columns of numbers representing uncertainty in population response, which may take the form of mean values.

Output file 1012 contains the resulting simulated outputs, and when combined with the inputs, the original hierarchical structure of the model may be inferred. However, such implicit determination of model structure from inputs/outputs is not generally within the ability of a non-expert Rather, the modeling expert must review and then present the results in a manner which allows an audience to recognize the model's hierarchical structure: here, behavior of the drug candidate is modeled based upon the three specific input variables. Such necessary conventional intervention by the human expert interferes with the audience's ability to meaningfully interact with the modeling results, and to develop intuition regarding the model's structure and operation.

Accordingly, FIG. 10B shows a schematic view of the automated operation of the DMX software to depict the output of the model to a non-expert audience in a meaningful way. Specifically, FIG. 10B shows a simplified schematic diagram illustrating the DMX software's generation of the metadata and binary files from the raw data files output by a model.

FIG. 10B is divided into two halves. The left half 1000 of FIG. 10B represents raw simulation output by a model in an arbitrary format that only implicitly reflects the hierarchical structure of the model. The right half 1002 of FIG. 10B represents the same data organized into the DMX format explicitly encoded to reflect the hierarchical structure of the model.

Starting at the upper left of FIG. 10B, simulation index file 1004 comprises a number of row vectors 1006 that describe unique modeling input scenarios. The row number 1008 associated with any given vector is used as an index to identify the corresponding column 1010 in the simulation output file 1012 represented in the lower left of FIG. 10B. Column 1010 comprises the distribution of numbers produced by the model when run through a simulation process utilizing the specific input scenario.

Review of the structure of raw data files 1004 and 1012 reveals that they explicitly include index information identifying treatment types and corresponding simulated results. These raw data files, however, reflect the hierarchical structure of the original model only implicitly. However, in order for a human audience member to understand the meaning of the simulated results, that person must be presented with the model structure explicitly. In other words, for a person to learn anything from a distribution of numbers, he or she must recognize that the distribution represents the expected effect for a particular endpoint, in a particular patient population, under a particular set of treatment conditions.

Software routines of the DMX data conversion modules 1014 receive files 1004 and 1012 as inputs, parses them, producing as output two new files 1016 and 1018. This saves the human modeling expert from having to construct an explicit representation of the model structure.

The DMX metadata file 1016 is a replacement for the simulation index file 1004. Index information contained in files 1004 and 1012 is extracted and utilized to encode the metadata file 1016. The data structure implicitly imparted to file 1004 by the hierarchical model organization, is thus transformed into an explicit, ordered XML tree structure.

DMX Binary file 1018 is a replacement for simulation output file 1012. Data contained in the original output file 1012 is converted in binary file 1018 into an n-dimensional hypercube structure. The geometry of this structure matches the tree structure of metadata file 1016. As a result of this transformation, the location in the binary file of simulation output corresponding to a given input vector, may be read from the model structure explicitly reflected in metadata file 1016.

Review of the structure of DMX data files 1016 and 1018 reveals that taken together, they locate treatment types and corresponding simulated results in a manner which explicitly reflects the hierarchical structure of the original model. Specifically, in this conceptual example limited to 3 dimensions for the convenience of communication, binary file 1018 comprises a structure having X-, Y-, and Z-axes corresponding to each of the input variables. In this manner, the original structure of the model may be readily discerned from the simulated data.

To summarize: prior to conversion by the DMX software, raw data output by the simulation model includes explicit index information that only implicitly reflects the hierarchical structure of the model. Following conversion by the DMX software, the simulation data is reorganized according to a metadata file encoded to explicitly reflect the hierarchical structure of the model.

The structure of the raw simulation output, and the structure of the DMX format data shown in FIG. 10B represent only particular examples of a large number of ways in which simulation output may be stored. In general, simulated numerical values output by the model will utilize some form of indexing to correlate model input scenarios with the respective output.

Use of the DMX software to alter the structure of the simulated output data from a raw format (where explicit index information only implicitly reflects model structure) to DMX format (where the binary file is organized according to metadata file explicitly reflecting model structure), adds value in at least a couple of essential ways.

First, the conversion process allows any arbitrary source of simulated data (i.e. model) to produce data which the DMX Software can parse and display. Addition of a new source of simulation data (i.e. the use of different statistical software to produce the raw simulation files) involves drafting additional conversion routines recognizing the file format and explicit index values presented by output of the different software, a relatively simple task. In this manner, the DMX system can be adapted and generalized to any arbitrary number of data sources, without requiring changes to the core DMX software.

Second, for reasons favoring the accuracy of modeling, the modeling expert may not generate raw simulation results in a compact or particularly efficient data structure. Conversion of such raw data into the DMX format in accordance with embodiments of the present invention, however, allows the simulation data to ultimately be presented by the DMX software to an audience in an orderly and compact format, regardless of the original raw format.

This second attribute of the DMX software is important because the order and compactness of the underlying data has a direct and positive affect on the utility and performance of the DMX software. The DMX conversion process thus frees the modeling expert to concentrate on producing as accurate simulation output as possible, without concern for convenience to the end user. The modeling expert may thus efficiently delegate to the DMX software, responsibility for automatically converting raw output into a compact and ordered data structure.

Third, parsing raw simulation data to extract the implicit model structure and then rendering it explicitly in the metadata file, saves the modeling expert the time and effort otherwise required to perform this work.

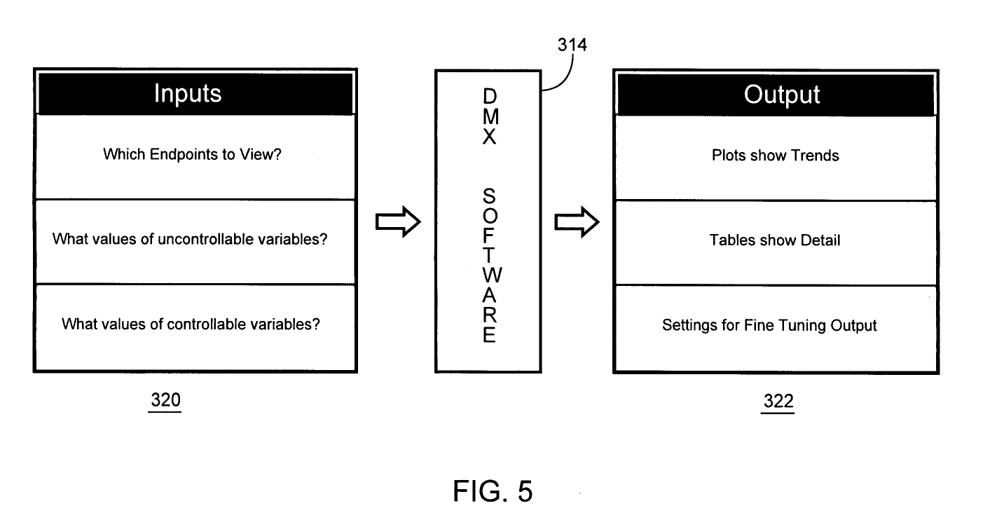

FIG. 5 shows a simplified schematic diagram of user inputs and outputs to one embodiment of a DMX software program in accordance with the present invention. Examples of inputs 320 to the DMX software 314 include but are not limited to 1) identification of endpoints to view, 2) values of uncontrollable variables, 3) values for controllable variables, and 4) modeling assumptions. Examples of outputs 322 from the DMX software 314 include but are not limited to 1) plots showing indicating trends in the data to be visualized by the software, 2) tables showing details of the data to be visualized by the software, and 3) settings for fine tuning data output by the software.

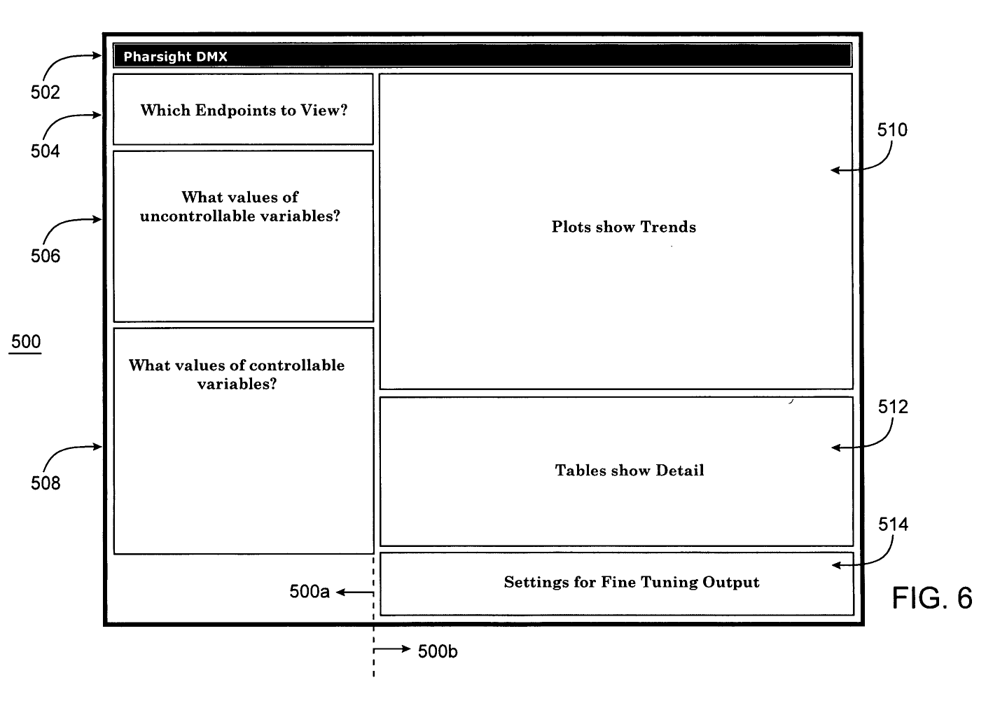

FIG. 6 shows a simplified depiction of generic fields of one embodiment of DMX software graphic user interface (GUI) screen 500. GUI screen 500 comprises title bar 502. Left hand portion 500a of screen 500 includes three input fields.

Top input field 504 indicates endpoints that are to be viewed. Endpoints are specifically the output of the model. Endpoints can be values measured directly in the clinical setting. For example, endpoints can be based upon physical signs observed in a patient. Examples of such measurable patient physical signs include, but are not limited to, blood pressure, heart-rate, presence of edema, body weight, and body temperature.

Endpoints can also be based upon symptoms of illness observed in a patient. Examples of such symptoms include, but are not limited to, shortness of breath, polyuria, fatigue, Erectile Dysfunction Score, and diarrhea. Patient signs and symptoms are described at length by Lynn Bickley in “Bates' Guide to Physical Examination & History Taking”, incorporated by reference herein for all purposes.

Endpoints can also be based upon the results of laboratory tests. Examples of such laboratory test results include, but are not limited to, Fasting Glucose, HbAlc, Triglycerides, Low Density Lipoproteins, and High Density Lipoproteins. Many other examples of Laboratory tests are described at http://www.labtestsonline.org/, incorporated by reference herein for all purposes.

Alternatively, endpoints can be values that are derived from clinical measures.

Examples of such endpoints that are derived from clinical measurements include, but are not limited to, absolute/percent/fractional change from baseline value of any endpoint measure, number/fraction/percent of patients staying below/reaching/exceeding a specific value of an endpoint measure, and percent/fraction of patients exhibiting an effect.

Further alternatively, an endpoint can also be the result of any arbitrary mathematical operation performed on one or more endpoints. For example, values of the same endpoint resulting from a difference in a controllable input can be contrasted to produce a measure of effect of that controllable input. For another type of derived value, values of the same endpoint resulting from a difference in an uncontrollable input can be contrasted to produce a measure of association with that uncontrollable input. Alternatively, different endpoints resulting from the same inputs can be combined (as in a weighted average) to produce a summary endpoint. Further alternatively, summary endpoints resulting from a difference in a controllable or uncontrollable input can be contrasted to produce a measure of the effect /association of the input on/with the summary endpoint.

In general, endpoints representing a clinical effect or which are directly derived from a clinical effect, are generally classified as either a benefit (positive) or side effect (negative), from taking the drug candidate. Valuation of the endpoint as positive or negative can, but need not, influence representation of the endpoint by the user interface of the DMX software.

Bottom input field 508 indicates values of controllable variables. Controllable variables are inputs that can be modified by human decision and are thus not determined by events outside human control. Controllable variables will in general be related to treatment, with the most obvious being choice of drug and dose. Other examples include, but are not limited to, frequency of drug administration and the formulation of the drug, (recommended) treatment drug, (recommended) treatment dose, (recommended) combination therapy drugs, (recommended) combination therapy doses, (recommended) frequency of drug administration, (actual) formulation of drug, (recommended) duration of drug administration, (recommended) subject diet regimen, and (recommended) subject exercise regimen.

Middle input field 506 indicates values of uncontrollable variables. Uncontrollable variables are inputs whose values cannot be controlled, or can only be partially controlled, by human decision. Uncontrollable variables reflect the “state of nature”, or the effect of events outside of human control. They may be observed, but cannot be controlled.

Uncontrollable variables are quite different than controllable inputs such as the choice of a treatment drug. Examples of uncontrollable inputs include, but are not limited to, the physical characteristics of a patient population such as sex, body weight/obesity, education, ethnicity, naivete to therapy, smoking, alcohol consumption, recreational drug use, actual compliance with prescribed therapy, baseline value of any biomarker used as endpoint, and assessments of disease progress (i.e. acute or mild).

Other variables determining model outcome are modeling assumptions. The human expert who builds the DMX model must make assumptions about the state of nature. The human expert responsible for building the DMX model may expose these assumptions as inputs, so that users of the DMX can visualize the effect of the assumptions on the output. For purposes of the instant patent application, the term “model” refers to a set of linked mathematical functions coupled with parameters quantifying functional relationships.

Examples of such model assumptions include, but are not limited to the form of the model, and the number, range, domain, and dimension of the linked mathematical functions. Other examples of assumptions include values of model parameters, utilization of specific published assumptions on the model form and/or parameters, utilization of a specific published model in its entirety (form and parameters), and utilization of specific published data to establish model form and/or parameters.

Right hand portion 500b of screen 500 includes three output displays. Top output display 510 is for data plots. Plots comprise one or more axes representing independent input, and one or more axes representing a corresponding output. One common specific plot format form has a single axis for independent input, and a single axis for dependent output. Another common specific plot format has a single access for independent input and two axes for dependent output.

In addition to the axes, plots may include one or more figures representing the trend of output along the dimension of the independent input, as well as possibly one or more figures representing the uncertainty in that output. The plots may also be decorated with figures partitioning output axes into ranges assigned subjective value ratings.

In addition to the independent input visualized on an axis, every plot will also reflect any number of background inputs (conditions) that locate the output represented on the plot in the multi-dimensional space described by the complete drug model. Specific embodiments of the DMX software in accordance with the present invention are capable of representing different sets of conditions (known as stratifications) by displaying a patterned collection of plots.

Such a patterned collection of plots is referred to as a “plot matrix”. In a plot matrix, the plots in a given row or column differ from each other in terms of the values of a particular input variable. This depiction permits the user to visualize how the output is affected as a single input changes, while all other remain constant.

Middle output display 512 is for data tables. The option to display data in tabular form provides a convenient format for the communication of exact numerical model output. In other words, tables will show exact numeric output corresponding to specific values of the currently selected, and plotted, independent and dependent variables.

The difference between tabular output and plot output is one of convenience. The output in either case is conceptually the same, with specific output values communicated more precisely in tabular than in plot form. On the other hand, plots are superior to tables in communicating data trends.

Bottom output display 514 is used to contain controls that let the user influence the output presentation, independent of model input. Controls allow the user to communicate preferences regarding graphic presentation of model output. Controls may be contrasted with inputs which allow the user change conditions under which output is to be generated.

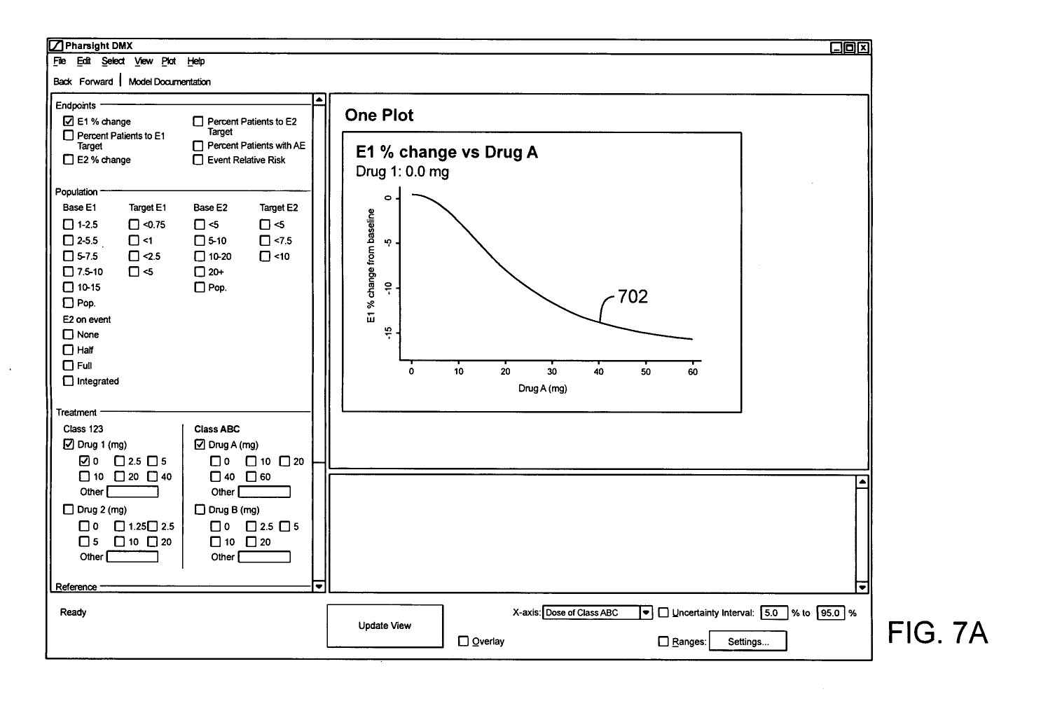

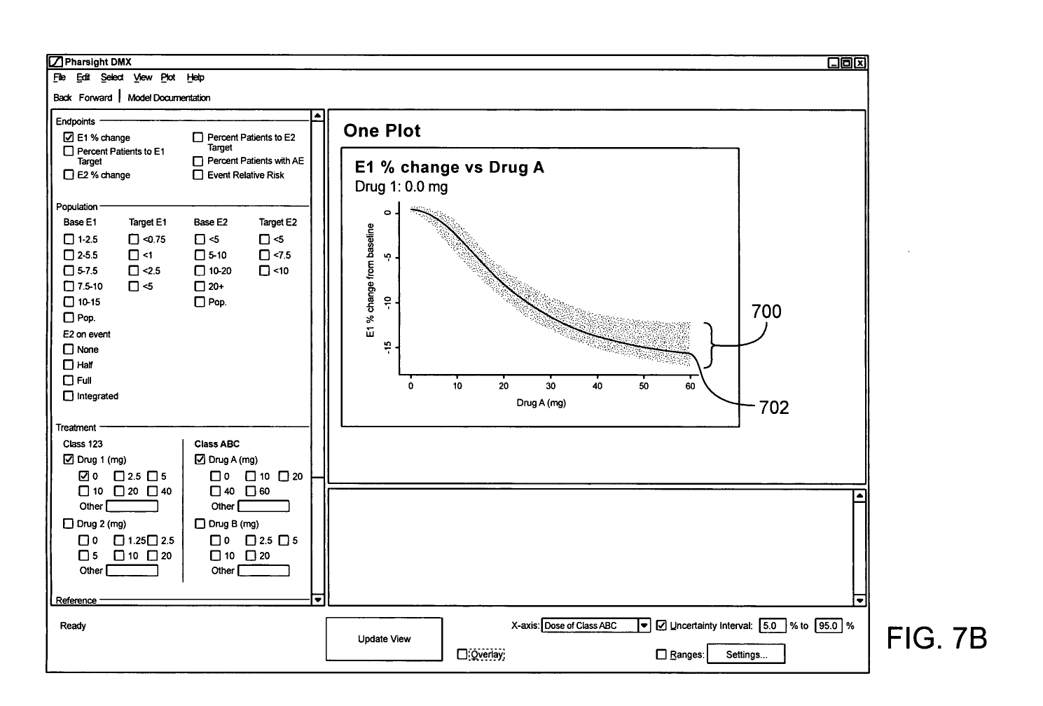

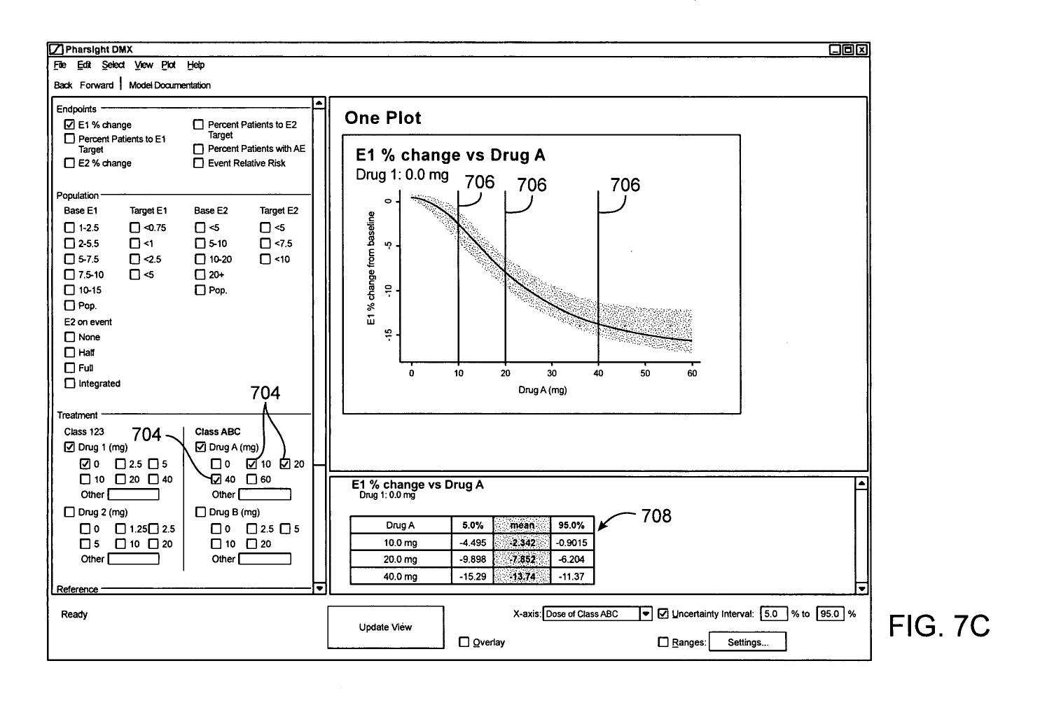

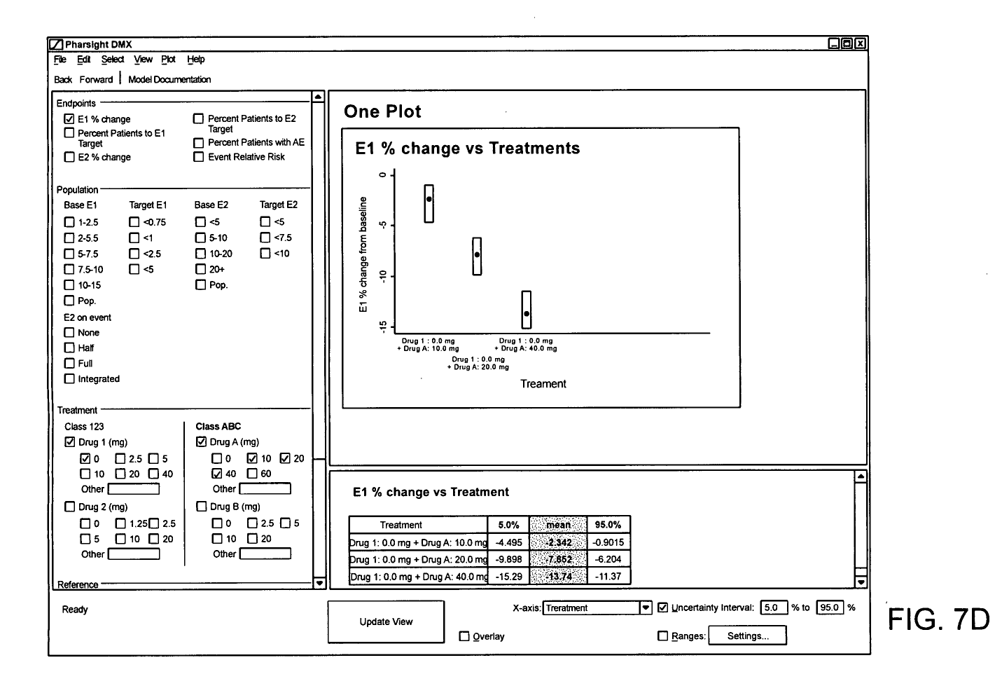

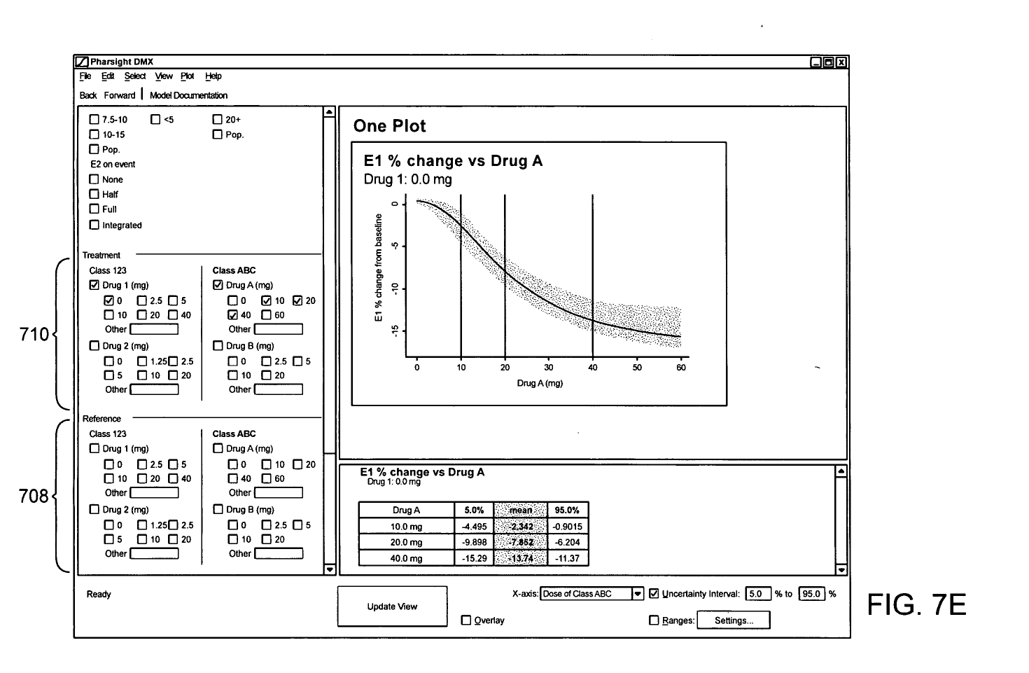

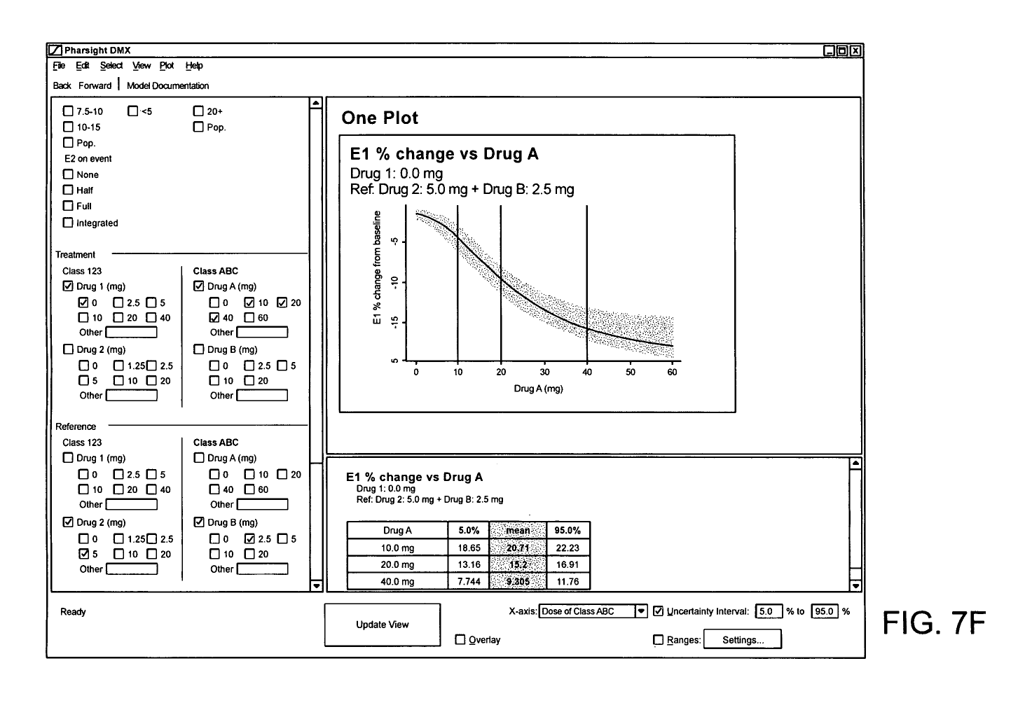

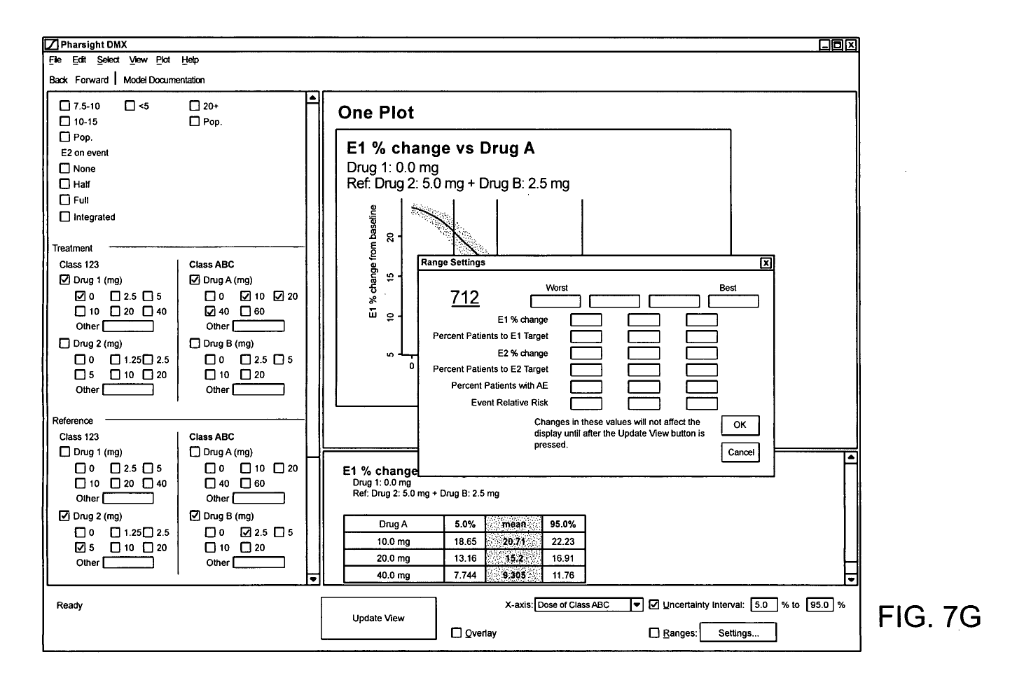

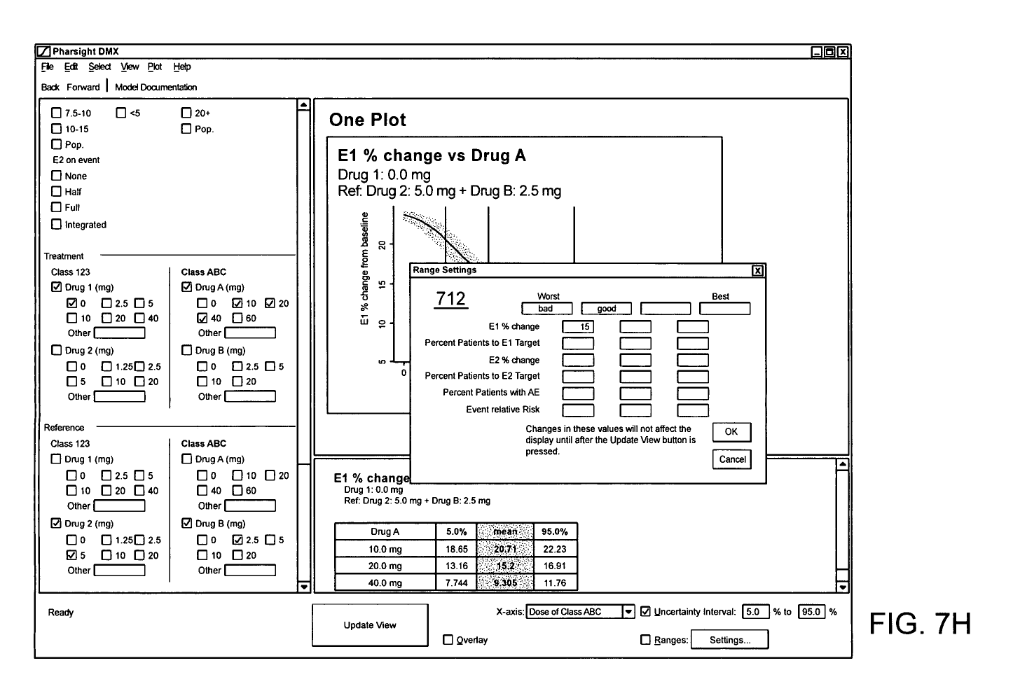

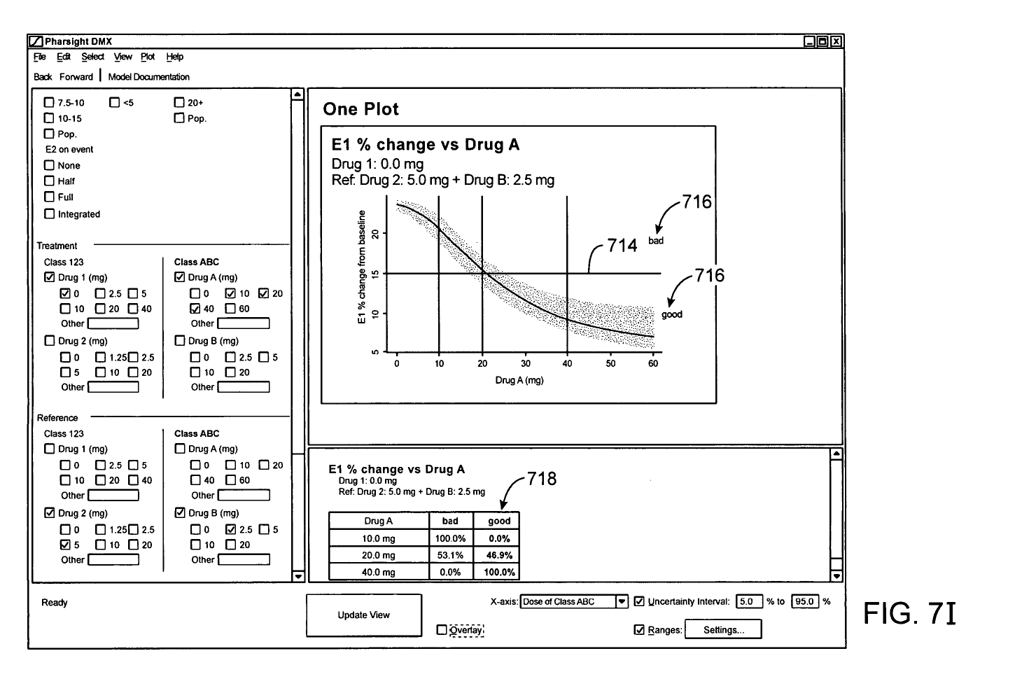

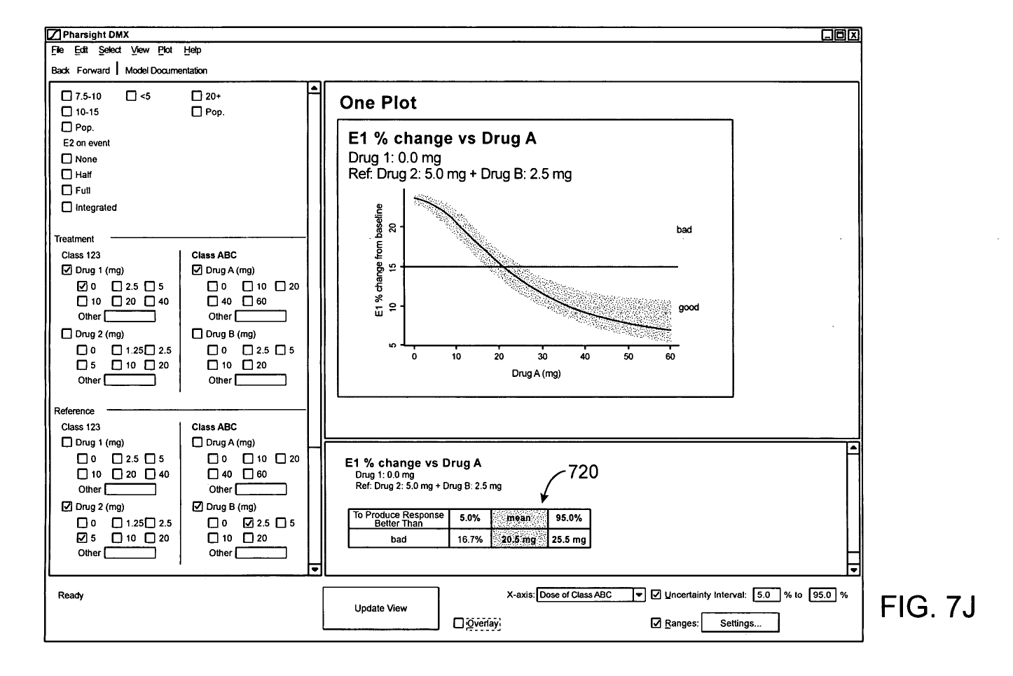

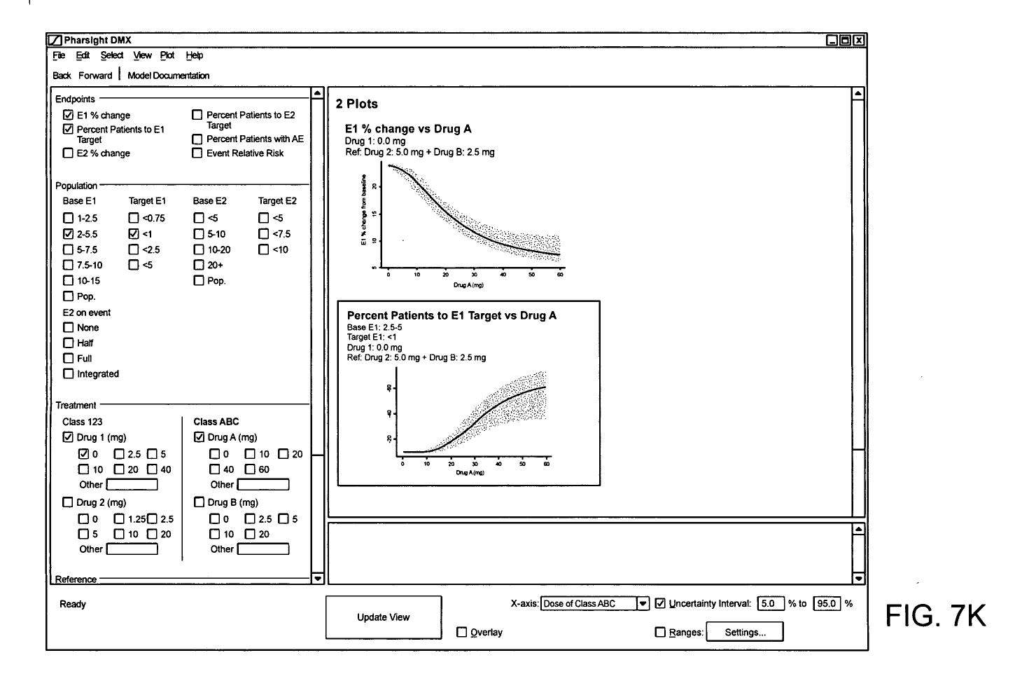

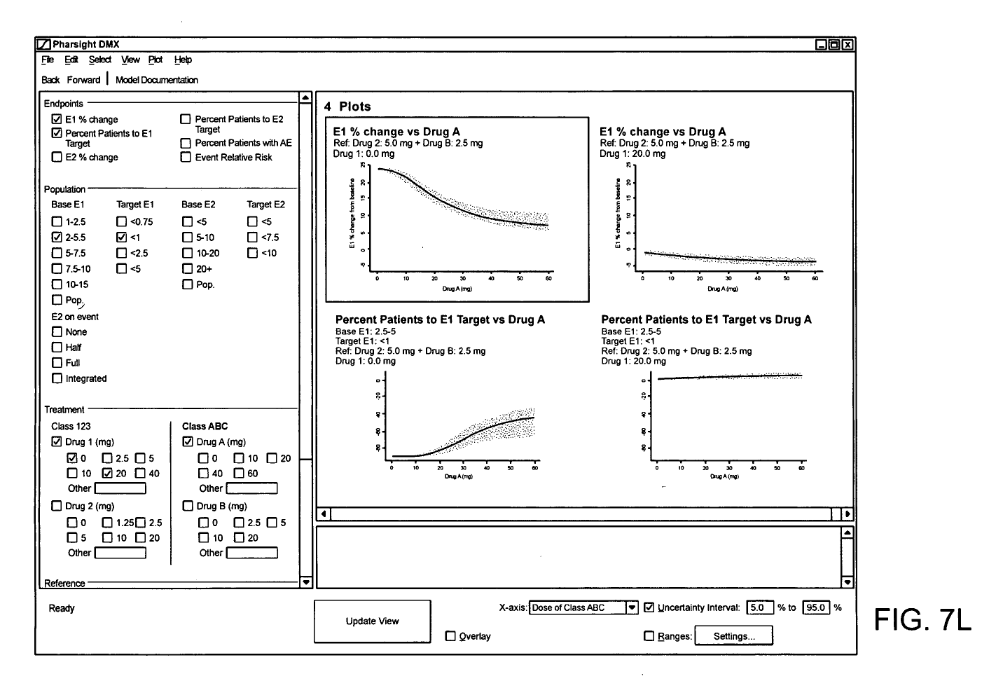

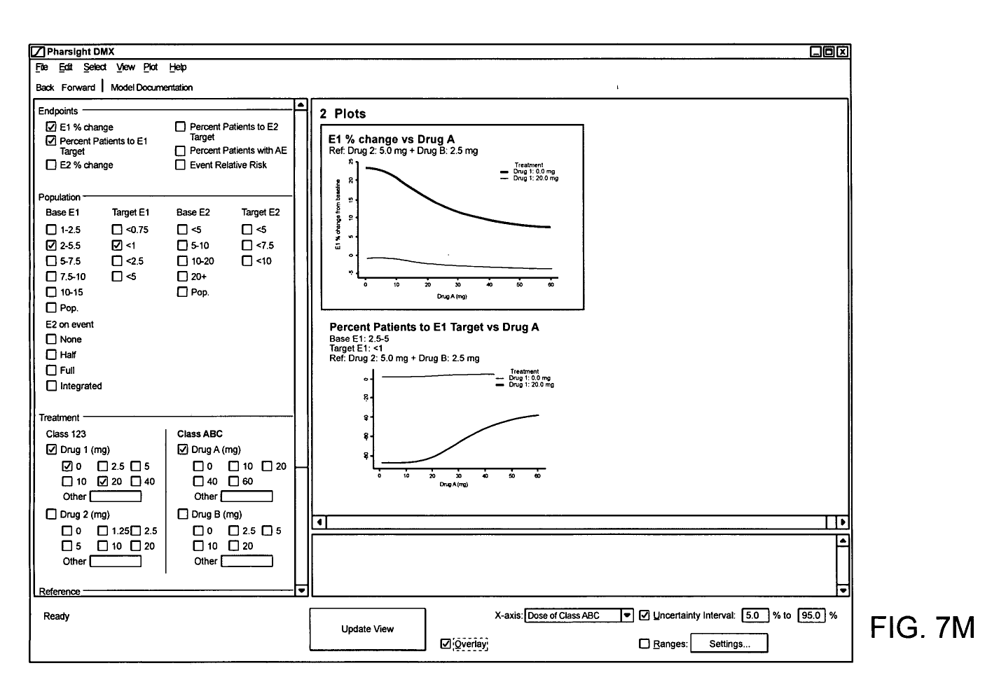

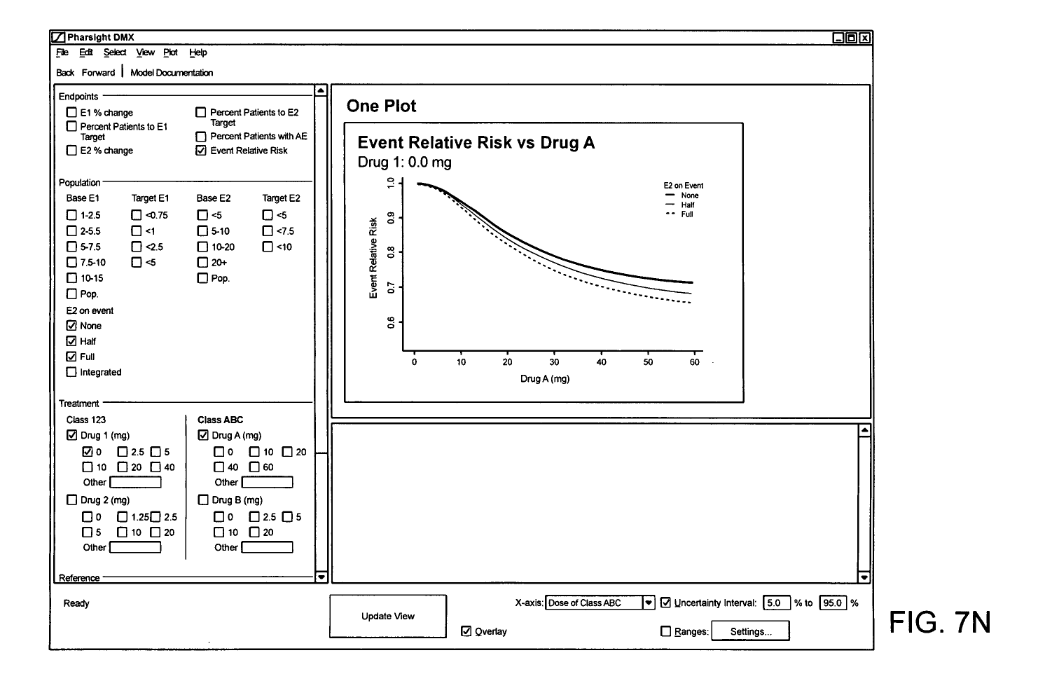

The generic GUI screen presented in FIG. 6 may be further understood by reviewing a specific example. FIGS. 7A-N show views presenting a simple but complete example of features of one specific graphic user interface of the DMX software.

In FIG. 7A, the GUI has been configured by the user to display the mean dose response for the endpoint E1 % change against a combination therapy of 0.0 mg of Drug 1 combined with Drug A. Because the Dose of Drug A has been selected as the independent variable, or x-axis variable, the resulting plot shows a continuous curve of expectation FIG. 702 representing the expected response as the Drug A dose varies from 0 to 60 mg.

FIG. 7B shows the GUI configured to display the same output as FIG. 7A, except that in FIG. 7B the user has selected display of an uncertainty interval 700 around the expectation FIG. 702. In this case the bounds of the uncertainty figure are set at 5% and 95%. This means that for any given dose of Drug A, there is a 90% chance the response will fall within the range marked by the uncertainty interval.

FIG. 7C is equivalent to FIG. 7B except that now the user has selected to highlight certain particular doses of Drug A—specifically 10, 20, and 40 mg. This selection is apparent in at least three places. In the controllable input zone, under Drug A, three checkboxes 704 are checked. In the plot, vertical lines 706 now appear at the selected doses. And finally, table 708 is now displayed in the output zone that lists the precise values for the 5%, expectation and 95% response for each of the highlighted doses. This table is termed a Clinical Effect Table. As described above, the output in the table is identical to the output in the plot, except that it is presented in a format more appropriate for communicating precise numerical values.

FIG. 7D shows the effect of selecting a different value for the independent variable. In this case, the value now selected is Treatment, which has caused the GUI to display a discrete plot of E1 % Change against Treatment, with the treatments being exactly those highlighted in FIG. 7C. This can be verified by noting the identical nature of the tables depicted in FIGS. 7C-D.

FIG. 7D illustrates that the GUI can support the presentation of different views in order to increase the convenience or ease of interpreting the underlying information. However, as can be seen by comparing FIG. 7D with FIG. 7C, changing the view changes the appearance, but not the content, of the displayed information.

FIG. 7E illustrates how a non-expert user can interact with and alter information presented by the DMX software. In FIG. 7E, the GUI is configured as in FIG. 7D, except that more controllable inputs are now visible. Specifically, a set of inputs 708 labeled “Reference” are now shown. These inputs 708 duplicate the overlying Treatment inputs 710, to allow the user to compare the effect of one treatment against another.

In FIG. 7F the user has selected and plotted a treatment comparison. Specifically, the displayed output compares the difference in effect on endpoint E1 % Change, of the combination of 0.0 mg Drug 1 and Drug A, versus the combination of 5 mg of Drug 2 and 2.5 mg of Drug B. The y-axis of the plot has shifted relative to FIG. 7E, and the values in the table have changed because the y-axis now represents the difference in effect on E1 % Change, of one therapy versus another. The plot of FIG. 7F is directly responsive to the common drug development question, “how much better (or worse) is one treatment than another?”

FIG. 7G shows a first screen of an interface method wherein the user is permitted to partition output by value. In FIG. 7G, dialog box is 712 shown overlaying the main interface screen. The purpose of this dialog is to allow the user to define and label different parts of the output range for each endpoint.

FIG. 7H shows a second screen of an interface method allowing the user to partition output by value. The user has entered data into the dialog box 712. Specifically, the user has partitioned the range of output for E1 % Change into two regions, good and bad. The user has thus defined the border between good and bad regions as occurring at a value of fifteen.

FIG. 71 shows the output screen resulting from the settings created in FIG. 7H. Horizontal line 714 and additional labels 716 now appear on the plot, graphically representing the value partition created.

In addition, the tabular output has changed form. As opposed to the Clinical Effect table displayed in previous Figures, table 718 of FIG. 7H is labeled as a “Range Table”. This Range Table shows, for each of the highlighted doses, the likelihood that the resulting effect will fall within each of the value partitions.

FIG. 7J shows the GUI configured to produce a third type of table: the Target Table 720. In FIG. 7J, the user has not highlighted any doses, but has maintained the value partition created in FIG. 7H. The DMX software calculates and displays in the GUI, the expected dose and uncertainty that would produce an effect exactly at the transition point between the value partitions. Note that as previously, the tables of FIGS. 71-J present the same information conveyed in a plot, but in a more precise format.

FIG. 7K shows the GUI configured to produce a matrix of plots where the conditioning dimension is endpoint. Specifically, two plots appear corresponding to the endpoints “E1 % Change” and “Percent Patients to E1 Target”. The controllable variable settings are identical for these two plots. The uncontrollable variable settings differ because the endpoint “Percent Patients to E1 Target” has been modeled in terms of additional inputs as compared to “E1 % Change”. In earlier Figures these inputs were shown as unavailable for selection, because until the user chose to display the endpoint “Percent Patients to E1 Target”, these inputs were not relevant to the output.

FIG. 7L shows the GUI configured to produce a plot matrix with two conditioning dimensions. The first dimension is endpoint, as in FIG. 7K. The second dimension is “Dose of Drug 1”. Specifically, plots in the left column show output when the dose of Drug 1 is 0.0 mg, and plots in the right column show output when the dose of Drug 1 is 20 mg.

FIG. 7M shows the GUI configured to produce the same output as FIG. 7L, except that the user has selected the plots be overlaid. As a result the four plots of FIG. 7L are condensed into two plots. In this case the row dimension of the matrix continues to be endpoint, but conditioning upon the dose of Drug 1 is now represented by two curves in each plot, rather than as two separate plots as in FIG. 7L.

FIG. 7N shows the GUI configured to visualize the effect of different assumptions upon the output. Specifically, the endpoint “Event Relative Risk” is plotted against “Dose of Drug A” for three different assumption values of “E2 on Event”. As can be observed, the different assumptions produce different outputs. While the input “E2 on Event” is not conceptually different than other uncontrollable inputs, FIG. 7N is included to highlight the ability of the present invention to render explicit certain assumptions, an important and useful feature.

As is apparent from reviewing FIGS. 3-7N and the accompanying discussion, the DMX software replaces the human expert as the intermediary between the model and the audience. By manipulating inputs to the graphic user interface component of the DMX software, non-expert members of the audience are readily able to obtain and display representations of relevant subsets of the large binary data file output by the model.

Embodiments of the DMX software and methodology in accordance with the present invention offer a number of important advantages over conventional modeling data interfaces based upon the presence of a mediating human expert. One such advantage is increased speed. Rather than being forced to explain proposed revisions to such a human expert, the audience can implement these changes directly in the software.

Another advantage offered by embodiments of the DMX software in accordance with the present invention is consistency. Specifically, the DMX software enables comparative expectations to be developed from disparate, non-uniform data sources, and permits those expectations to rigorously be assigned specific, statistically valid risk levels.

Through a rigorous and formal process of integration, use of the DMX software fosters the creation of new quantitative knowledge regarding the likely behavior of a compound, especially in relation to possible competitors. Such an approach stands in contrast to conventional model interfaces requiring human experts to perform interpretations based upon intuition and experience that are not readily quantifiable or reproducible between different human experts, or even between different instances of interpretation by the same human expert.

Still another advantage of the DMX software is to offer a common set of visual representations for evaluation of a multitude of drug candidate compounds by all audience members across different institutions. Specifically, the DMX interface provides at least three visual innovations.

First, the DMX software shows the dose response of a compound as simple pictures across all relevant clinical endpoints. Second, for every response, the DMX software provides an easy way to visualize both an expectation and an uncertainty on the same picture. Finally, the DMX software allows users to create simple visual representations illustrating the relative response of a compound, as compared to potential competing products.

The expression of complex modeling results utilizing the simple visual language offered by the DMX software has both short- and long-term benefits. In the short term, this visual representation means that any audience member, technical or not, can easily learn to understand modeling and simulation results.

In the longer term, audience understanding a standardized visual presentation of information by the DMX software regarding one drug program can rapidly translate into understanding of similarly presented results coming from other drug programs. The implication is that the DMX software can create a general “language” by which drug development communities can share information rapidly, clearly and without ambiguity.

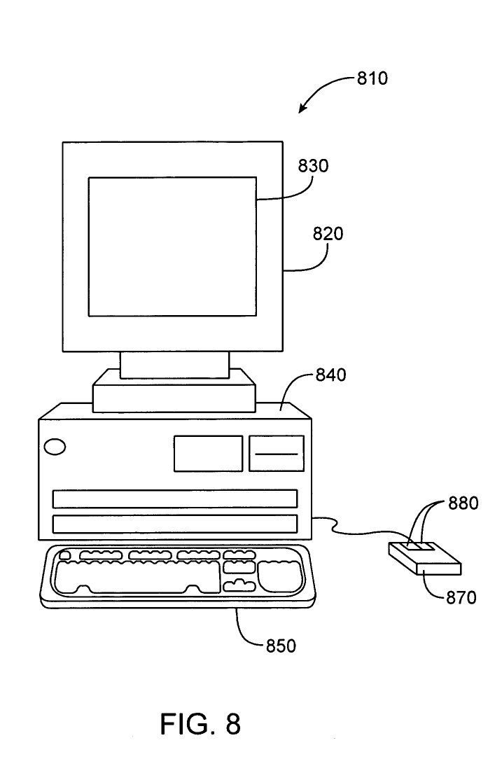

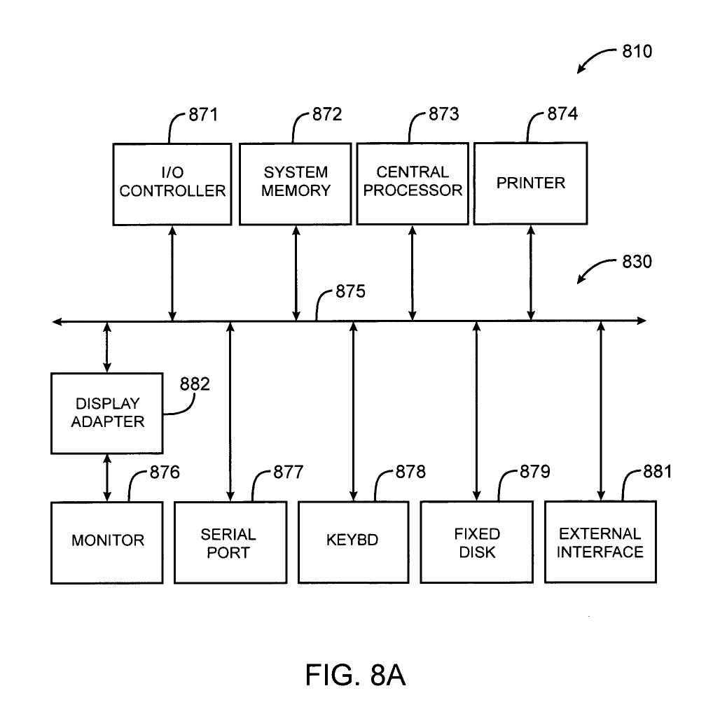

As described in detail above, embodiments of drug discovery methods in accordance with embodiments of the present invention are particularly suited for implementation in conjunction with a computer. FIG. 8 is a simplified diagram of a computing device for processing information according to an embodiment of the present invention. This diagram is merely an example which should not limit the scope of the claims herein. One of ordinary skill in the art would recognize many other variations, modifications, and alternatives. Embodiments according to the present invention can be implemented in a single application program such as a browser, or can be implemented as multiple programs in a distributed computing environment, such as a workstation, personal computer or a remote terminal in a client server relationship.

FIG. 8 shows computer system 810 including display device 820, display screen 830, cabinet 840, keyboard 850, and mouse 870. Mouse 870 and keyboard 850 are representative “user input devices.” Mouse 870 includes buttons 880 for selection of buttons on a graphical user interface device. Other examples of user input devices are a touch screen, light pen, track ball, data glove, microphone, and so forth. FIG. 8 is representative of but one type of system for embodying the present invention. It will be readily apparent to one of ordinary skill in the art that many system types and configurations are suitable for use in conjunction with the present invention. In a preferred embodiment, computer system 810 includes a Pentium™ class based computer, running Windows™ NT operating system by Microsoft Corporation. However, the apparatus is easily adapted to other operating systems and architectures by those of ordinary skill in the art without departing from the scope of the present invention.

As noted, mouse 870 can have one or more buttons such as buttons 880. Cabinet 840 houses familiar computer components such as disk drives, a processor, storage device, etc. Storage devices include, but are not limited to, disk drives, magnetic tape, solid state memory, bubble memory, etc. Cabinet 840 can include additional hardware such as input/output (I/O) interface cards for connecting computer system 810 to external devices external storage, other computers or additional peripherals, further described below.

FIG. 8A is an illustration of basic subsystems in computer system 810 of FIG. 8. This diagram is merely an illustration and should not limit the scope of the claims herein. One of ordinary skill in the art will recognize other variations, modifications, and alternatives. In certain embodiments, the subsystems are interconnected via a system bus 875. Additional subsystems such as a printer 874, keyboard 878, fixed disk 879, monitor 876, which is coupled to display adapter 882, and others are shown. Peripherals and input/output (I/O) devices, which couple to I/O controller 871, can be connected to the computer system by any number of means known in the art, such as serial port 877. For example, serial port 877 can be used to connect the computer system to a modem 881, which in turn connects to a wide area network such as the Internet, a mouse input device, or a scanner. The interconnection via system bus allows central processor 873 to communicate with each subsystem and to control the execution of instructions from system memory 872 or the fixed disk 879, as well as the exchange of information between subsystems. Other arrangements of subsystems and interconnections are readily achievable by those of ordinary skill in the art. System memory, and the fixed disk are examples of tangible media for storage of computer programs, other types of tangible media include floppy disks, removable hard disks, optical storage media such as CD-ROMS and bar codes, and semiconductor memories such as flash memory, read-only-memories (ROM), and battery backed memory.

While the above is a full description of the specific embodiments, various modifications, alternative constructions and equivalents may be used. Therefore, the above description and illustrations should not be taken as limiting the scope of the present invention.

1. A method of representing performance of a drug candidate, the method comprising:
receiving raw data generated by a model of drug candidate behavior, the raw data comprising index information, treatment scenario input information types, and corresponding output performance information types;
extracting the index information from the raw data;
referencing the extracted index information to generate a metadata file, a structure of the metadata file explicitly reflecting a hierarchical structure of the model;
referencing the metadata file to convert the raw data file into a binary file, the metadata file explicitly identifying locations of treatment scenario information types and the output performance information types within the binary file;
generating a user interface from the metadata file, the interface comprising a menu of input variables;
presenting the menu to a user;
receiving a user-selected input at the interface;
causing the interface to reference the metadata file and the binary file to identify a subset of the binary file relevant to the user-selected input; and
presenting the data subset in one of a select type of presentation formats at the interface.

2. The method of claim 1 wherein the data subset represents a clinical effect.

3. The method of claim 1 wherein the data subset represents a likelihood of a clinical effect lying within a range of user-defined value.

4. The method of claim 1 wherein the data subset represents a value of an independent variable required for a clinical effect to one of attain, exceed, and equal a user-defined value.

5. The method of claim 1 wherein the data subset represents a value of an independent variable required for a clinical effect to fall one of within, above, and below a user-defined range of values.

6. The method of claim 1 wherein the presentation format comprises a table.

7. The method of claim 1 wherein the presentation format comprises a matrix of tables.

8. The method of claim 1 wherein the presentation format comprises a plot.

9. The method of claim 1 wherein the presentation format comprises a matrix of plots.

10. The method of claim 1 wherein the data subset represents a contrast between output corresponding to two controllable variable input scenarios.

11. The method of claim 10 wherein the data subset represents a contrast between output corresponding to a first controllable variable input scenario featuring the drug candidate, and a second controllable variable input scenario featuring a competitor of the drug candidate.

12. The method of claim 10 wherein the contrast represents one of a difference, a ratio, and a log ratio.

13. The method of claim 1 wherein the menu of input variables is selected from the group consisting of an endpoint, a controllable variable, and an uncontrollable variable.

14. The method of claim 13 wherein endpoint is based upon a clinically measured value.

15. The method of claim 13 wherein the controllable variable is selected from the group comprising drug candidate identity, drug candidate dose, frequency of administration of drug candidate, and formulation of the drug candidate.

16. The method of claim 13 wherein the uncontrollable variable comprises a patient attribute selected from the group consisting of age, gender, body weight, and disease state.

17. The method of claim 13 wherein the uncontrollable variable comprises a model assumption.

18. The method of claim 1 wherein the raw data comprises a file organized according to explicit index values, and the metadata file encodes the explicit index values into a structure.

19. The method of claim 18 wherein the raw data comprises multiple files.

20. The method of claim 18 wherein the raw data is converted into the single binary file organized to match the encoded structure.

21. The method of claim 18 wherein the raw data is converted into multiple binary files organized to match the encoded structure.

22. The method of claim 18 wherein the explicit index values are encoded into an ordered tree structure.

23. The method of claim 22 wherein the binary file comprises an n-dimensional structure having a geometry matching the tree structure.

24. The method of claim 1 wherein the menu comprises text from the Metadata file.

25. The method of claim 1 further comprising drafting an additional conversion routine configured to recognize the raw data structure, and to transform the raw data into a standard metadata file format.

26. A computer system comprising a processor and a memory storing code to operate the processor, the code comprising, a parser module configured to receive raw data output by a model of drug candidate behavior, and to generate a metadata file encoding outputs and related inputs of the model based upon index information extracted from the raw data;
a data transfer module configured to convert the raw data into a binary file organized to match a structure encoded in the metadata file; and
a graphic user interface configured to present a menu of input variables to a user, to receive inputs selected by the user, to reference the metadata file and the binary file to identify a subset of the binary file relevant to the selected inputs, and to present the data subset in one of a select type of presentation format.

27. The computer system of claim 26 wherein the raw data comprises:
an index file having row vectors including a row number, the row vectors describing unique modeling input scenarios, and
a simulation output file comprising columns of number distributions produced by the model when run through a simulation process utilizing the specific input scenario, a column number corresponding to the row number; and wherein,
the metadata file is organized according to a tree structure, and the binary file is organized into an n-dimensional structure whose geometry matches the tree structure.

28. The computer system of claim 26 wherein the parser module further comprises a conversion routine configured to recognize a format of the model, and to transform the raw data into a standard format of the metadata file.
